(19) United States
(12) Patent Application Publication
(10) Pub. No.: US 2025/0115675 A1
(43) Pub. Date: Apr. 10, 2025

(54) CODON-OPTIMIZED NUCLEIC ACIDS ENCODING OCRELIZUMAB

(71) Applicant: AMGEN INC., Thousand Oaks, CA (US)

(72) Inventors: Rachana Vengarai, Moorpark, CA (US); Huong LE, Newbury Park, CA (US)

(73) Assignee: AMGEN INC., Thousand Oaks, CA (US)

(21) Appl. No.: 18/836,051

(22) PCT Filed: Feb. 6, 2023
(86) PCT No.: PCT/US2023/062044

§ 371 Date: Aug. 6, 2024

Related U.S. Application Data
(60) Provisional application No. 63/307,688, filed on Feb. 8, 2022.

(51) Int. Cl. C07K 16/28

(52) U.S. Cl. CPC C07K 16/2887; C07K 2317/14; C07K 2317/24

(57) ABSTRACT

The present invention relates to recombinant proteins that have been codon optimized for recombinant expression.

### CROSS-REFERENCE TO RELATED APPLICATIONS

This application claims the benefit of U.S. Provisional Application No. 63/307,688, filed Feb. 8, 2022, and incorporated herein by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 54,960 XML Document file named “10010-WO01-SEC_SeqListing”; created on Feb. 6, 2023.

### FIELD OF THE INVENTION

The present invention relates to recombinant proteins that have been codon optimized for recombinant expression.

### BACKGROUND

The CD20 antigen (also called human B-lymphocyte-restricted differentiation antigen, Bp35) is a hydrophobic transmembrane protein with a molecular weight of approximately 35 kD located on pre-B and mature B lymphocytes (Valentine et al. J. Biol. Chem. 264 (19): 11282-11287 (1989); and Einfeld et al. EMBO J. 7 (3): 711-717 (1988)). CD20 regulates an early step(s) in the activation process for cell cycle initiation and differentiation and possibly functions as a calcium ion channel (Tedder et al. J. Cell. Biochem. 14D: 195 (1990)). CD20 is the target of the monoclonal antibodies including rituximab, ocrelizumab, obinutuzumab, ofatumumab, ibritumomab tiuxetan, tositumomab, and ublituximab, for the treatment of B cell lymphomas, leukemias, and B cell-mediated autoimmune diseases.

B cells play a central role in the pathogenesis of multiple sclerosis (MS). They are involved in the activation of pro-inflammatory T cells, secretion of pro-inflammatory cytokines and production of autoantibodies directed against myelin. Ocrelizumab, sold under the brand name Ocrevus®, is a pharmaceutical agent for the treatment of MS. It is a humanized anti-CD20 monoclonal IgG1 antibody that selectively depletes B cells. Ocrelizumab has been shown to slow down clinically observed and imaging-based progression of relapsing forms of MS, as well as the primary progressive form of the disease.

There is a need to develop methods for efficient recombinant production of recombinant proteins, such as ocrelizumab, in mammalian cell lines, especially the industrially relevant Chinese hamster ovary (CHO) cells.

### SUMMARY

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1. A recombinant nucleic acid encoding an anti-CD20 antibody, wherein (i) said antibody comprises the light chain (LC) complementarity-determining region (CDR) 1, CDR2, and CDR3 of SEQ ID NO:3, and the heavy chain (HC) CDR1, CDR2, and CDR3 of SEQ ID NO:4; and (ii) wherein said nucleic acid comprises codons that are optimized for Chinese hamster ovary (CHO) cell expression.

E2. The nucleic acid of E1, wherein said heavy chain CDR1, CDR2, CDR3, and light chain CDR1, CDR2, and CDR3 are defined by Kabat as shown in the Sequence Table.

E3. The nucleic acid of E1 or E2, wherein said antibody comprises a heavy chain variable region (VH) that is at least 90%, at least 91%, at least 92%, at least 93%, at least 94%, at least 95%, at least 96%, at least 97%, at least 98%, at least 99%, or 100% identical to SEQ ID NO. 4.

E4. The nucleic acid of any one of E1-E3, wherein said antibody comprises a light chain variable region (VL) that is at least 90%, at least 91%, at least 92%, at least 93%, at least 94%, at least 95%, at least 96%, at least 97%, at least 98%, at least 99%, or 100% identical to SEQ ID NO. 3.

E5. The nucleic acid of any one of E1-E4, wherein said antibody comprises a heavy chain that is at least 90%, at least 91%, at least 92%, at least 93%, at least 94%, at least 95%, at least 96%, at least 97%, at least 98%, at least 99%, or 100% identical to SEQ ID NO. 2.

E6. The nucleic acid of any one of E1-E5, wherein said antibody comprises a light chain that is at least 90%, at least 91%, at least 92%, at least 93%, at least 94%, at least 95%, at least 96%, at least 97%, at least 98%, at least 99%, or 100% identical to SEQ ID NO. 1.

E7. The nucleic acid of any one of E1-E6, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs. 7, 9, 11, 13, 15, 17, 19, 21, 23, 25, 27, and 29.

E8. The nucleic acid of any one of E1-E7, comprising a light chain VL coding sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOS. 7, 11, 15, 19, 23, and 27.

E9. The nucleic acid of any one of E1-E8, comprising a heavy chain VH coding sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs. 9, 13, 17, 21, 25, and 29.

E10. The nucleic acid of any one of E1-E9, comprising a light chain VL coding sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOS. 7, 11, 15, 19, 23, and 27; and a heavy chain VH coding sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs. 9, 13, 17, 21, 25, and 29.

E11. The nucleic acid of any one of E1-E10, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 7.

E12. The nucleic acid of any one of E1-E11, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 9.

E13. The nucleic acid of any one of E1-E12, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 7, and a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 9.

E14. The nucleic acid of any one of E1-E10, comprising a sequence that is at least 80%, at least 81%, at least 82%, at least 83%, at least 84%, at least 85%, at least 86%, at least 87%, at least 88%, at least least 97%, at least 98%, at least 99%, or 100% identical to SEQ ID NO. 11.

E15. The nucleic acid of any one of E1-E10 and E14, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical SEQ ID NO. 13.

E16. The nucleic acid of any one of E1-E10 and E14-E15, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 11, and a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 13.

E17. The nucleic acid of any one of E1-E10, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 15.

E18. The nucleic acid of any one of E1-E10 and E17, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 17.

E19. The nucleic acid of any one of E1-E10 and E17-E18, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 15, and a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 17.

E20. The nucleic acid of any one of E1-E10, comprising a sequence that is at least 80%, at least 81%, at least 82%, at least 83%, at least 84%, at least 85%, at least 86%, at least 87%, at least 88%, at least least 97%, at least 98%, at least 99%, or 100% identical to SEQ ID NO. 19.

E21. The nucleic acid of any one of E1-E10 and E20, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 21.

E22. The nucleic acid of any one of E1-E10 and E20-E21, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 19, and a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 21.

E23. The nucleic acid of any one of E1-E10, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 23.

E24. The nucleic acid of any one of E1-E10 and E23, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 25.

E25. The nucleic acid of any one of E1-E10 and E23-E24, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 23, and a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 25.

E26. The nucleic acid of any one of E1-E10, comprising a sequence that is at least 80%, at least 81%, at least 82%, at least 83%, at least 84%, at least 85%, at least 86%, at least 87%, at least 88%, at least least 97%, at least 98%, at least 99%, or 100% identical to SEQ ID NO. 27.

E27. The nucleic acid of any one of E1-E10 and E26 comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 29.

E28. The nucleic acid of any one of E1-E10 and E26-E27, comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 27, and a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO. 29.

E29. The nucleic acid of any one of E1-E28, wherein said nucleic acid hybridizes to any one of SEQ ID NOs. 7, 9, 11, 13, 15, 17, 19, 21, 23, 25, 27, and 29 under moderately stringent conditions.

E30. The nucleic acid of any one of E1-E29, wherein said nucleic acid comprises a VL coding sequence that hybridizes to any one of SEQ ID NOs. 7, 11, 15, 19, 23, and 27 under moderately stringent conditions.

E31. The nucleic acid of any one of E1-E30, wherein said nucleic acid comprises a VH coding sequence that hybridizes to any one of SEQ ID NOs. 9, 13, 17, 21, 25, and 29 under moderately stringent conditions.

E32. The nucleic acid of any one of E1-E31, wherein said nucleic acid comprises a VL coding sequence that hybridizes to any one of SEQ ID NOs. 7, 11, 15, 19, 23, and 27 under moderately stringent conditions, and a VH coding sequence that hybridizes to any one of SEQ ID NOs. 9, 13, 17, 21, 25, and 29 under moderately stringent conditions.

E33. The nucleic acid of any one of E1-E32, wherein said nucleic acid comprises a VL coding sequence that hybridizes to SEQ ID NO:7 under moderately stringent conditions, and a VH coding sequence that hybridizes to SEQ ID NO:9 under moderately stringent conditions.

E34. The nucleic acid of any one of E1-E32, wherein said nucleic acid comprises a VL coding sequence that hybridizes to SEQ ID NO:11 under moderately stringent conditions, and a VH coding sequence that hybridizes to SEQ ID NO:13 under moderately stringent conditions.

E35. The nucleic acid of any one of E1-E32, wherein said nucleic acid comprises a VL coding sequence that hybridizes to SEQ ID NO: 15 under moderately stringent conditions, and a VH coding sequence that hybridizes to SEQ ID NO: 17 under moderately stringent conditions.

E36. The nucleic acid of any one of E1-E32, wherein said nucleic acid comprises a VL coding sequence that hybridizes to SEQ ID NO:19 under moderately stringent conditions, and a VH coding sequence that hybridizes to SEQ ID NO:21 under moderately stringent conditions.

E37. The nucleic acid of any one of E1-E32, wherein said nucleic acid comprises a VL coding sequence that hybridizes to SEQ ID NO:23 under moderately stringent conditions, and a VH coding sequence that hybridizes to SEQ ID NO:25 under moderately stringent conditions

E38. The nucleic acid of any one of E1-E32, wherein said nucleic acid comprises a VL coding sequence that hybridizes to SEQ ID NO:27 under moderately stringent conditions, and a VH coding sequence that hybridizes to SEQ ID NO:29 under moderately stringent conditions.

E39. The nucleic acid of E29-E38, wherein said moderately stringent conditions comprise prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

E40. The nucleic acid of any one of E1-E39, wherein said nucleic acid hybridizes to any one of SEQ ID NOs. 7, 9, 11, 13, 15, 17, 19, 21, 23, 25, 27, and 29 under highly stringent conditions.

E41. The nucleic acid of any one of E1-E40, wherein said nucleic acid comprises a VL coding sequence that hybridizes to any one of SEQ ID NOs. 7, 11, 15, 19, 23, and 27 under highly stringent conditions.

E42. The nucleic acid of any one of E1-E41, wherein said nucleic acid comprises a VH coding sequence that hybridizes to any one of SEQ ID NOs. 9, 13, 17, 21, 25, and 29 under highly stringent conditions.

E43. The nucleic acid of any one of E1-E42, wherein said nucleic acid comprises a VL coding sequence that hybridizes to any one of SEQ ID NOs. 7, 11, 15, 19, 23, and 27 under highly stringent conditions, and a VH coding sequence that hybridizes to any one of SEQ ID NOs. 9, 13, 17, 21, 25, and 29 under highly stringent conditions.

E44. The nucleic acid of any one of E1-E43, wherein said nucleic acid comprises a VL coding sequence that hybridizes to SEQ ID NO:7 under highly stringent conditions, and a VH coding sequence that hybridizes to SEQ ID NO:9 under highly stringent conditions.

E45. The nucleic acid of any one of E1-E43, wherein said nucleic acid comprises a VL coding sequence that hybridizes to SEQ ID NO:11 under highly stringent conditions, and a VH coding sequence that hybridizes to SEQ ID NO:13 under highly stringent conditions.

E46. The nucleic acid of any one of E1-E43, wherein said nucleic acid comprises a VL coding sequence that hybridizes to SEQ ID NO:15 under highly stringent conditions, and a VH coding sequence that hybridizes to SEQ ID NO: 17 under highly stringent conditions.

E47. The nucleic acid of any one of E1-E43, wherein said nucleic acid comprises a VL coding sequence that hybridizes to SEQ ID NO: 19 under highly stringent conditions, and a VH coding sequence that hybridizes to SEQ ID NO:21 under highly stringent conditions.

E48. The nucleic acid of any one of E1-E43, wherein said nucleic acid comprises a VL coding sequence that hybridizes to SEQ ID NO:23 under highly stringent conditions, and a VH coding sequence that hybridizes to SEQ ID NO:25 under highly stringent conditions

E49. The nucleic acid of any one of E1-E43, wherein said nucleic acid comprises a VL coding sequence that hybridizes to SEQ ID NO:27 under highly stringent conditions, and a VH coding sequence that hybridizes to SEQ ID NO:29 under highly stringent conditions.

E50. The nucleic acid of E40-E49, wherein said highly stringent conditions comprise: (1) low ionic strength and high temperature for washing; (2) use of a denaturing agent during hybridization, or (3) use of 50% formamide, 5×SSC, 50 mM sodium phosphate, 0.1% sodium pyrophosphate, 5× Denhardt's solution, sonicated salmon sperm DNA, 0.1% SDS, and 10% dextran sulfate at 42° C., with washes at 42° C. in 0.2×SSC and 50% formamide at 55° C., followed by a high-stringency wash of 0.1×SSC containing EDTA at 55° C.

E51. A vector comprising the nucleic acid of any one of E1-E50.

E52. The vector of E51, wherein said nucleic acid is operably linked to a promoter.

E53. A host cell comprising the nucleic acid of any one of E1-E50.

E54. A host cell comprising the vector of E51 or E52.

E55. The host cell of E53 or E54, wherein said cell is a mammalian cell.

E56. The host cell of E55, wherein said host cell is a CHO cell.

E57. A method of making an anti-CD20 antibody, or antigen-binding fragment thereof, comprising culturing the host cell of any one of E53-E56 under a condition wherein said antibody or antigen-binding fragment is expressed by said host cell.

E58. The method of E57, further comprising isolating said antibody or antigen-binding fragment thereof.

### BRIEF DESCRIPTION OF THE DRAWINGS

FIG. 1 is a plasmid map illustrating the vectors used in the Examples.

FIG. 2 shows the recovery profiles of the different codon sets used in the Examples.

FIG. 3 shows the growth profiles observed for the different codon sets during fed-batch production.

FIG. 4 shows the viability profiles observed for the different codon sets during fed-batch production.

FIG. 5 shows the product titers observed among the different codon sets.

FIGS. 6A-6B show the alignment of the different codon sets used in the Examples. FIG. 6A is an alignment of heavy chain variable region, and FIG. 6B is an alignment of light chain variable region.

### DETAILED DESCRIPTION

### 1. Condon Optimization

Efficient recombinant production of antibodies in mammalian cell lines, especially the industrially relevant Chinese hamster ovary (CHO) cells, is an important area of biotechnology research. The bottleneck at protein translation has been recognized as an important issue in the design of heterologous gene for recombinant expression. The poor translation of heterologous protein may be due to the difference in codon usage bias between the expression host and recombinant gene. As a result of random mutation and selection pressure, different organisms may have evolved to utilize the synonymous codons with disparate frequencies.

Ocrelizumab is a humanized anti-CD20 IgG1 antibody. When expressing a foreign gene in a particular host organism (e.g. CHO cell), the differences in codon bias can hinder the protein translation process in a manner whereby the host is unable to efficiently translate the rare codons that may occur frequently in the recombinant gene. As such, coding sequence re-design via codon optimization may be required to adapt the foreign gene for efficient heterologous expression.

Among the various parameters considered for such DNA sequence design, individual codon usage (ICU) has been implicated as one of the crucial factors affecting mRNA translational efficiency. Further, influence of codon pair usage, also known as codon context (CC), can also affect the level of protein expression. Usage of sequential codon-pairs is non-random and unique to each species.

As disclosed and exemplified herein, the amino acid sequence of Ocrelizumab was reversely translated to nucleotide sequence. Initially, the expression level in a CHO cell line was unsatisfactory. The poor expression level of ocrelizumab in CHO host cells was solved by codon optimization. Among the different algorithms tested, three codon optimized sets showed 3-fold to 9-fold titer improvement from the original CHO pools.

The amino acid sequences of ocrelizumab light and heavy chains are publicly available and provided in the Sequence Table. Due to codon degeneracy, multiple nucleotide sequences can be obtained from the same amino acid sequence, and further sequence selection and/or codon optimization may be needed. Factors affecting mRNA traffic, stability and expression should be considered. For example, codons may need to be altered to change the overall mRNA AT (AU)-content, to minimize or remove all potential splice sites, and to alter any other inhibitory sequences and signals affecting the stability and processing of mRNA such as runs of A or T/U nucleotides, AATAAA, ATTTA and closely related variant sequences, known to negatively affect mRNA stability. Exemplary codon optimization methods can be found, e.g., in U.S. Pat. Nos. 6,794,498; 6,414, 132; 6,291,664; 5,972,596; and 5,965,726. For example, a relatively more A/T-rich codon of a particular amino acid may be replaced with a relatively more G/C-rich codon encoding the same amino acid

Generally, changes to the nucleotide bases or codons do not alter the amino acid sequence of the protein. The changes are based upon the degeneracy of the genetic code, utilizing an alternative codon for an identical amino acid, as summarized in Table 1. In certain embodiments, it will be desirable to alter one or more codons to encode a similar amino acid residue rather than an identical amino acid residue. Applicable conservative substitutions of coded amino acid residues are described above.

TABLE 1
Inverse table for the standard genetic code

| Amino | | | Amino | | |
|---|---|---|---|---|---|
| acid | DNA codons | Compressed | acid | DNA codons | Compressed |
| Ala, A | GCU, GCC, GCA, | GCN | Ile, I | AUU, AUC, AUA | AUH |
| | GCG | | | | |
| Arg, R | CGU, CGC, CGA, | CGN, AGR; | Leu, L | CUU, CUC, CUA, | CUN, UUR; |
| | CGG; AGA, AGG | or | | CUG; UUA, UUG | or |
| | | CGY, MGR | | | CUY, YUR |
| Asn, N | AAU, AAC | AAY | Lys, K | AAA, AAG | AAR |
| Asp, D | GAU, GAC | GAY | Met, M | AUG | |
| Asn or | AAU, AAC; GAU, | RAY | Phe, F | UUU, UUC | UUY |
| Asp, B | GAC | | | | |
| Cys, C | UGU, UGC | UGY | Pro, P | CCU, CCC, CCA, | CON |
| | | | | CCG | |
| Gln, Q | CAA, CAG | CAR | Ser, S | UCU, UCC, UCA, | UCN, AGY |
| | | | | UCG; AGU, AGC | |
| Glu, E | GAA, GAG | GAR | Thr, T | ACU, ACC, ACA, | ACN |
| | | | | ACG | |
| Gln or | CAA, CAG; GAA, | SAR | Trp, W | UGG | |
| Glu, Z | GAG | | | | |
| Gly, G | GGU, GGC, GGA, | GGN | Tyr, Y | UAU, UAC | UAY |
| | GGG | | | | |
| His, H | CAU, CAC | CAY | Val, V | GUU, GUC, GUA, | GUN |
| | | | | GUG | |

| | | | | |
|---|---|---|---|---|
| START | AUG | STOP | UAA, UGA, UAG | URA, UAR |

Depending on the number of changes introduced, the codon optimized nucleic acid sequences of the present invention can be conveniently made as completely synthetic sequences. Techniques for constructing synthetic nucleic acid sequences encoding a protein or synthetic gene sequences are well known in the art. Synthetic gene sequences can be commercially purchased through any of a number of service companies, including DNA 2.0 (Menlo Park, CA), Geneart (Toronto, Ontario, Canada), CODA Genomics (Irvine, CA), and GenScript, Corporation (Piscataway, NJ). Alternatively, codon changes can be introduced using techniques well known in the art. The modifications also can be carried out, for example, by site-specific in vitro mutagenesis or by PCR or by any other genetic engineering methods known in art which are suitable for specifically changing a nucleic acid sequence. In vitro mutagenesis protocols are described, for example, in In Vitro Mutagenesis Protocols, Braman, ed., 2002, Humana Press, and in Sankaranarayanan, Protocols in Mutagenesis, 2001, Elsevier Science Ltd.

Nucleic acid sequences that improve the expression level of anti-CD20 antibody can be constructed by altering select codons throughout the coding sequence, or by altering codons at the 5′-end, the 3′-end, or within a middle subsequence. It is not necessary that every codon be altered, but that a sufficient number of codons are altered so that the expression (i.e., transcription and/or translation) level can be increased. In some embodiments, the codon-optimized sequence increases the expression of anti-CD20 antibody by at least 10%, at least 20%, at least 30%, at least 40%, at least 50%, at least 60%, at least 70%, at least 80%, or at least 90% as compared to that of the original coding sequence, under substantially the same expression conditions. In some embodiments, the codon-optimized sequence increases the expression of anti-CD20 antibody by at least 1-fold, at least 2-fold, at least 3-fold, at least 4-fold, at least 5-fold, at least 6-fold, at least 7-fold, at least 8-fold, at least 9-fold, or at least 10-fold as compared to that of the original coding sequence, under substantially the same expression conditions. Expression can be detected over time or at a designated endpoint, using techniques known to those in the art, for example, using gel electrophoresis or binding assays (e.g., ELISA, immunohistochemistry).

In some embodiments, the coding sequence also comprises a signal peptide. Exemplary signal peptides include those from tissue plasminogen activator (tPA) protein, growth hormone, GM-CSF, and immunoglobulin proteins. Exemplary signal peptide sequences are provided in the Sequence Table, and also are known in the art (see, Lo, et ah, Protein Eng. (1998) 11:495 and Gen Bank Accession Nos. Z75389 and D14633). During translation, signal peptide is cleaved and is absent from mature immunoglobulins. Exemplary nucleic acid sequences encoding a signal peptide are also shown in the Sequence Table.

Accordingly, the invention provides a recombinant nucleic acid encoding an anti-CD20 antibody, wherein (i) said antibody comprises the light chain (LC) complementarity-determining region (CDR) 1,CDR2, and CDR3 of SEQ ID NO:3, and the heavy chain (HC) CDR1, CDR2, and CDR3 of SEQ ID NO:4;and (ii) wherein said nucleic acid comprises codons that are optimized for Chinese hamster ovary (CHO) cell expression. In some embodiments, the heavy chain CDR1, CDR2, CDR3, and light chain CDR1,CDR2, and CDR3 are defined by Kabat as shown in the Sequence Table.

In some embodiments, the VH and VL domains, or antigen-binding portion thereof, or full-length HC or LC, are encoded by separate nucleic acid molecules. Alternatively, both VH and VL, or antigen- binding portion thereof, or HC and LC, are encoded by a single nucleic acid molecule.

In some embodiments, the anti-CD20 antibody comprises a heavy chain variable region (VH) that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO. 4. In some embodiments, the anti-CD20 antibody comprises a light chain variable region (VL) that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO. 3.

In some embodiments, the anti-CD20 antibody comprises a heavy chain that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO. 2. In some embodiments, the anti-CD20 antibody comprises a light chain that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO. 1

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 7.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 9.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO.11.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 13.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 15.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 17.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 19.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 21.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 23.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 25.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 27.

In some embodiments, the codon-optimized nucleic acid molecule comprises a sequence that is least 60%, least 65%, least 70%, least 75%, least 80%, least 81%, least 82%, least 83%, least 84%, least 85%, least 86%, least 87%, least 88%, least 89%, least 90%, at least 91%, at least 92%, at least 93%, at least 94%, at least 95%, at least 96%, at least 97%, at least 98%, at least 99%, or 100% identical to SEQ ID NO. 29.

Two nucleic acid or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, WI), using default parameters. This program embodies several alignment schemes described in the following references: Dayhoff, M. O., 1978, A model of evolutionary change in proteins-Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5:151-153; Myers, E. W. and Muller W., 1988, CABIOS 4:1 1-17; Robinson, E. D., 1971, Comb. Theor. 1 1:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

In some embodiments, the codon-optimized nucleic acid molecule hybridizes to any one of SEQ ID NOs. 7, 9, 11, 13, 15, 17, 19, 21, 23, 25, 27, and 29 under moderately stringent conditions. In some embodiments, the codon-optimized nucleic acid molecule hybridizes to any one of SEQ ID NOs. 7, 9, 11, 13, 15, 17, 19, 21, 23, 25, 27, and 29 under highly stringent conditions.

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 ig/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

Nucleic acid sequences complementary to any of the sequences disclosed herein are also encompassed by the present disclosure. The nucleic acid may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-oding sequences may, but need not, be present within a polynucleotide of the present disclosure, and a polynucleotide may, but need not, be linked to other molecules and/or support materials.

The nucleic acid disclosed herein can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For example, PCR allows reproduction of DNA sequences. PCR technology is well known in the art and is described in U.S. Patent Nos. 4,683, 195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, for example.

### 2. Vectors and Host Cells

Once a codon optimized nucleic acid sequence has been constructed, it can be cloned into a cloning vector before subjecting to further manipulations for insertion into one or more expression vectors. Manipulations of recombinant nucleic acid sequences, including recombinant modifications and purification, can be carried out using procedures well known in the art. Such procedures have been published, for example, in Sambrook and Russell, Molecular Cloning: A Laboratory Manual, 2000, Cold Spring Harbor Laboratory Press and Current Protocols in Molecular Biology, Ausubel, et al., eds., 1987-2006, John Wiley & Sons.

Suitable cloning vectors may be constructed according to standard techniques, or may be selected from a large number of cloning vectors available in the art. While the cloning vector selected may vary according to the host cell intended to be used, useful cloning vectors will generally have the ability to self- replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

An anti-CD20 can be recombinantly expressed from an expression vector comprising the codon optimized nucleic acid sequences disclosed herein. Expression vectors generally are replicable nucleic acid constructs that contain an antibody coding sequence disclosed herein. It is implied that an expression vector must be replicable in the host cells either as episomes or as an integral part of the chromosomal DNA.

The expression vectors may have an expression cassette that will express an anti-CD20 antibody in a suitable host cell, such as a mammalian cell. The heavy chain and light chain of the antibody can be expressed from the same or multiple vectors. The heavy chain and light chain of the antibody can be expressed from the same vector from one or multiple expression cassettes (e.g., a single expression cassette with an internal ribosome entry site; or a double expression cassette using two promoters and two polyA sites). Within each expression cassette, sequences encoding the anti-CD20 antibody can be operably linked to expression regulating sequences. Exemplary expression control sequences include appropriate transcription initiation, termination, promoter and enhancer sequences; efficient RNA processing signals such as splicing and polyadenylation signals; sequences that stabilize cytoplasmic mRNA; sequences that promote RNA export (e.g., a constitutive transport element (CTE), a RNA transport element (RTE), or combinations thereof, including RTEm26CTE); sequences that enhance translation efficiency (e.g., Kozak consensus sequence); sequences that enhance protein stability; and when desired, sequences that enhance protein secretion.

The expression vector can also express a selectable marker. Selectable markers are well known in the art, and can include, for example, proteins that confer resistance to an antibiotic, fluorescent proteins, antibody epitopes, etc. Exemplified markers that confer antibiotic resistance include sequences encoding β-lactamases (against β-lactams including penicillin, ampicillin, carbenicillin), or sequences encoding resistance to tetracyclines, aminoglycosides (e.g., kanamycin, neomycin), etc. Exemplified fluorescent proteins include green fluorescent protein, yellow fluorescent protein and red fluorescent protein.

Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors comprising the nucleic acid disclosed herein can be introduced into the host cell by any of a number of appropriate means, including by direct uptake, endocytosis, electroporation, F-mating, transfection (such as those employing calcium chloride, rubidium chloride, calcium phosphate, DEAE-dextran, or other substances); microprojectile bombardment; lipofection; and infection (e.g., where the vector is an infectious agent such as vaccinia virus). The choice of introducing vectors or nucleic acids will often depend on features of the host cell. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Cloning vectors can be introduced into any suitable host cells. Exemplary host cells include an E. coli cell, a yeast cell, an insect cell, a simian COS cell, a Chinese hamster ovary (CHO) cell, a Human embryonic kidney (HEK) 293 cell, an Sp2.0 cell, or a myeloma cell where the cell does not otherwise produce an immunoglobulin protein, among many cells well-known in the art. Preferred host cell for an expression vector is a CHO cell.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purpose

### EXAMPLES

### Example 1. Host Cell Screening

During the initial screening, three types of CHO host cells, including a CHO-MGAT line and a CHO-GS KO line, were evaluated with 54 pools in total (data not shown). Low antibody titers were observed across all hosts in the initial screening phase. Increased selection stringency using MTX/MSX resulted in increased in expression levels in CHO-MGAT host but not in CHO-GS KO host (data not shown). Thus, codon optimization strategy was used to improve the antibody titers.

### Example 2. Ocrelizumab Nucleotide Sequence Generation From Amino Acid Sequence

The amino acid sequence of the Ocrelizumab was reverse transcribed to generate various nucleotide sequences encoding for the same original amino acid sequence. This reverse transcription process involves inputting the amino acid sequence into codon optimization platforms which use different codon usage tables to generate multiple nucleotide sequences with the highest theoretical expression levels in the selected host. Three codon optimization platforms were used for codon optimization of Ocrelizumab nucleotide sequences, and they were referred to as Algorithm 1, Algorithm 2, and Algorithm 3, respectively. These three algorithms produced six codon sets altogether.

Codon optimization for ocrelizumab heavy chain (HC) and light chain (LC) was performed only on the amino acids within the variable regions. The constant regions were not modified from the original backbone for IgG1, which is based on heavy chain VH3 and light chain VK1 sequences. Four sets of HC and LC pairs were generated. Sets 1-3 were based on the three platforms described above. The fourth set was a “hybrid” sequence that was generated as follows. Two other monoclonal antibody sequences that share high sequence similarity with ocrelizumab were analyzed. The codons in ocrelizumab that are different from the other two sequences were identified and replaced with most commonly used codons in the other two sequences.

### Example 3. Plasmid Generation

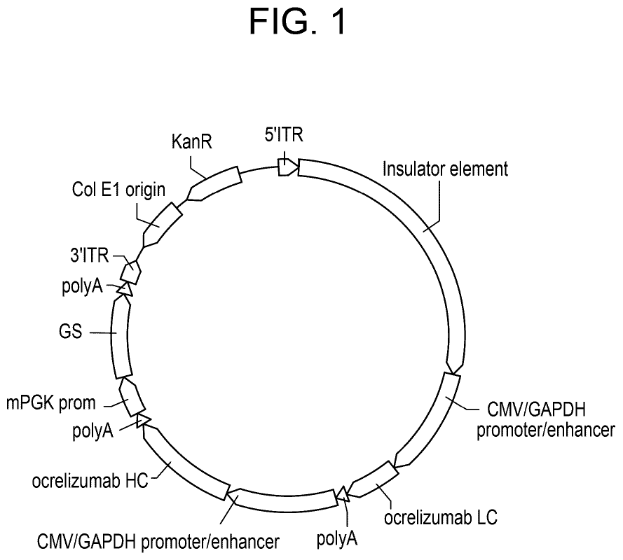

The coding sequences of the ocrelizumab LC and HC were inserted in the pPBGS4.1 plasmid backbone using golden gate cloning. Briefly, the LC with polyA fragment, the 2 CMV/GAPDH promoter/enhancer fragments, the HC fragment, the mPGK promoter and GS fragment, and the polyA to insulator fragment were uni-directionally assembled as shown in FIG. 1 using combinations of overhang sequences to facilitate golden gate cloning.

### Example 4. Transfection of Plasmid Into CHO Host

A glutamine synthetase knockout (GS KO) clonal cell host, derived from the CHO-K1 parental host, was used for generating stable pools expressing ocrelizumab. Host cells were passaged at a seeding density of 0.4-0.3×10⁶ cells/mL every 3-4 days in a proprietary DMEM-F12-based media in shake flasks at 120 rpm, 36° C. and 5% CO₂. Twenty-four hours before transfection, the host cells were seeded at 1×10⁶ cells/mL to ensure the cells would be in exponential growth phase at transfection.

Stable pools expressing orelizumab were generated using a Gene Pulser XCell (BioRad Laboratories; Hercules, CA) following the manufacturer's protocol. Duplicate transfections were performed for each of the six codon sets. Briefly, 20 ug of pPBGS4.1 plasmid in combination with 5 ug of a piggybac transposase were electroporated into 20×10⁶ host cells. The transfected cells were recovered in 20 mL of growth media in 50 mL spin tubes at 225 rpm, 36° C. and 5% CO₂.

### Example 5. Selection and Recovery

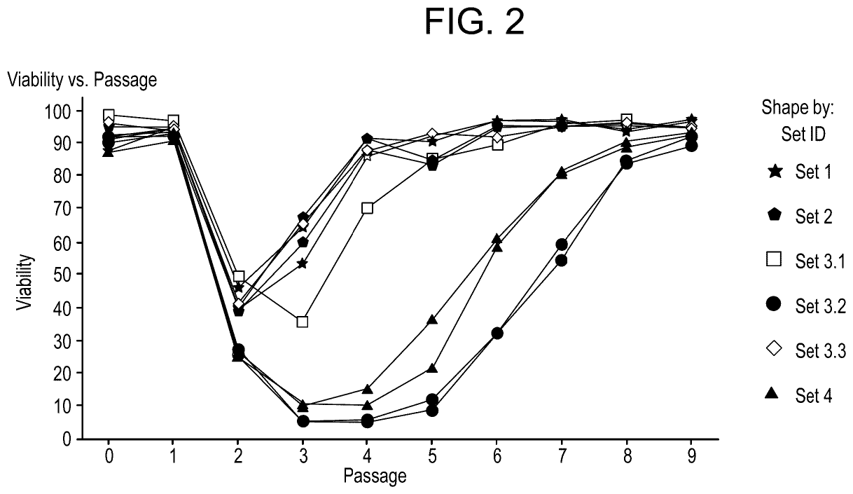

Seventy-two hours post transfection, the cells were spun down and transferred into selection media without Glutamine and with 12.5 uM of methionine sulfoximine (MSX). The cells were passaged at seeding densities around 1-2×10⁶ cells/mL every 3-4 days until viability reached over 90%, when the seeding density was reduced to 0.4-0.3×10⁶ cells/mL. FIG. 2 shows that similar recovery profiles were observed for all six codon sets.

### Example 6. Fed-Batch Production

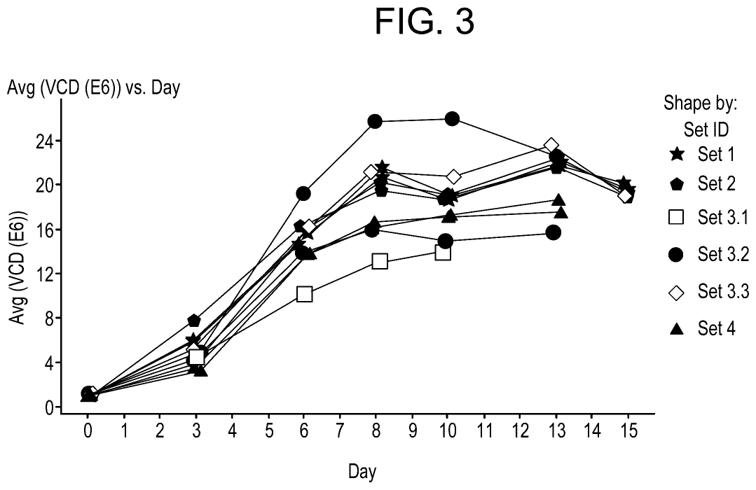

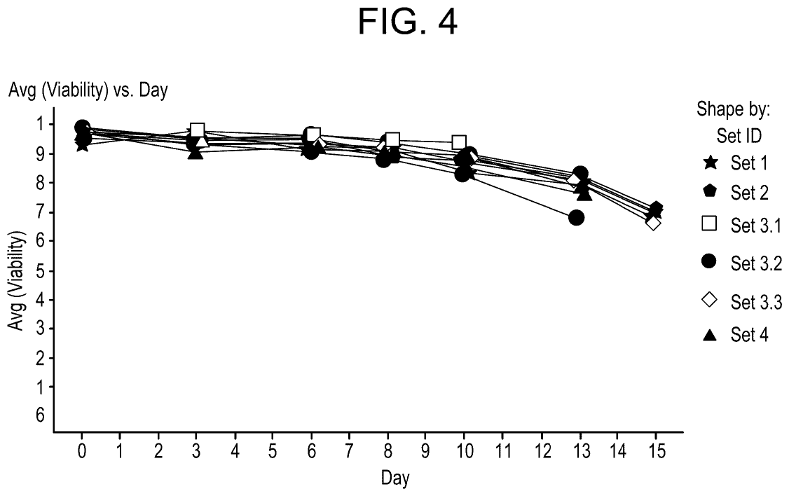

Fully recovered cells were inoculated fed-batch production at 1×10⁶ cells/mL in a proprietary basal media. The cultures were supplemented with Amgen proprietary feeds on day 3, 6, 8, 10, and 13 and harvested on day 15. Cell count and viability were determined using a Vi-Cell BLU cell viability analyzer (Beckman Coulter, Brea, CA). Product titer in the supernatant was measured by affinity POROS Protein A high performance liquid chromatography (HPLC) (Applied Biosystems, Carlsbad, CA). FIG. 3 shows that similar growth profiles were observed for all six codon sets during fed-batch production. FIG. 4 shows that similar viability profiles were observed for all six codon sets during fed-batch production.

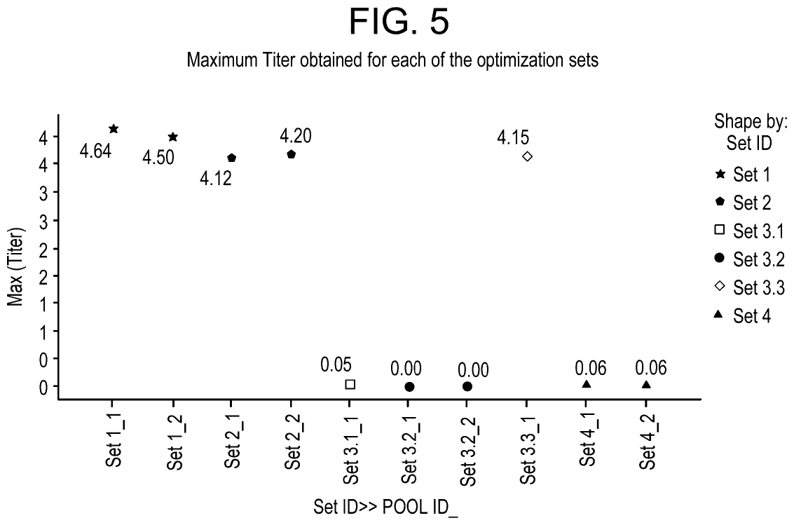

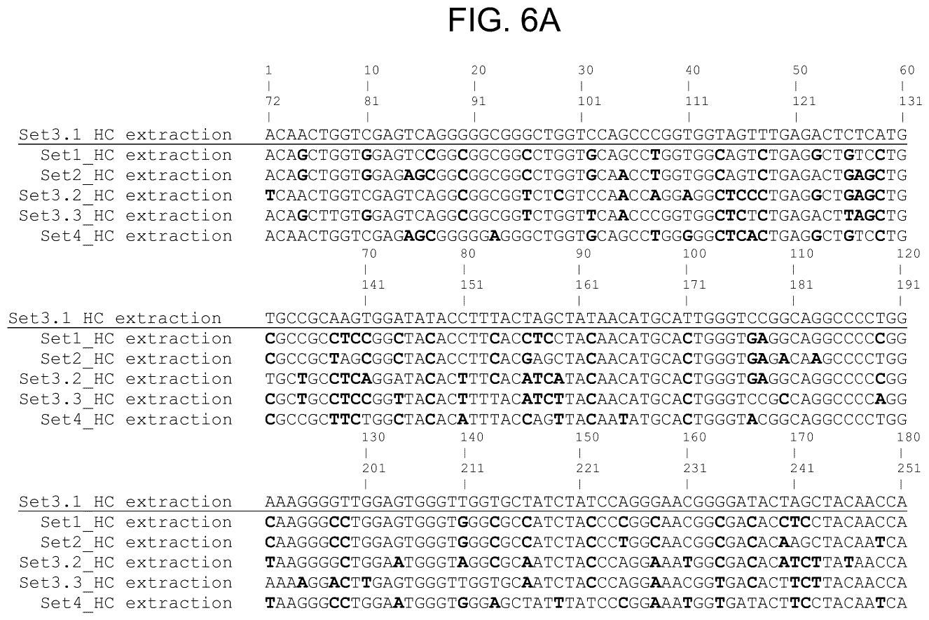

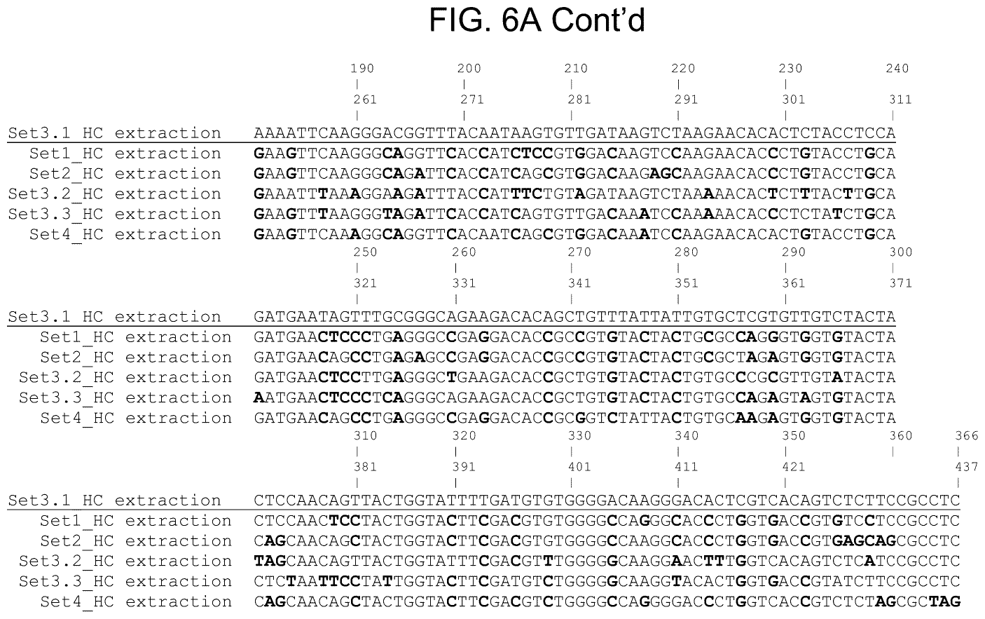

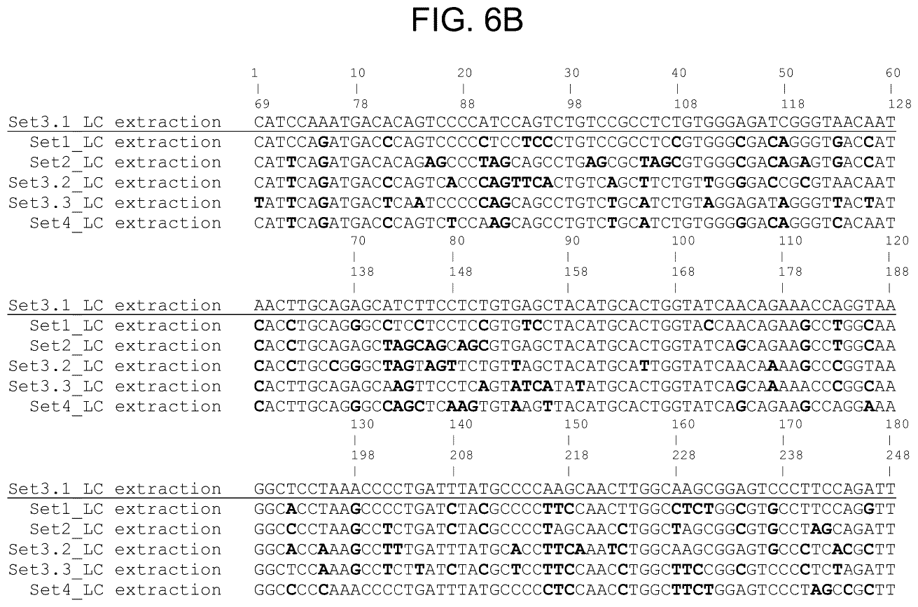

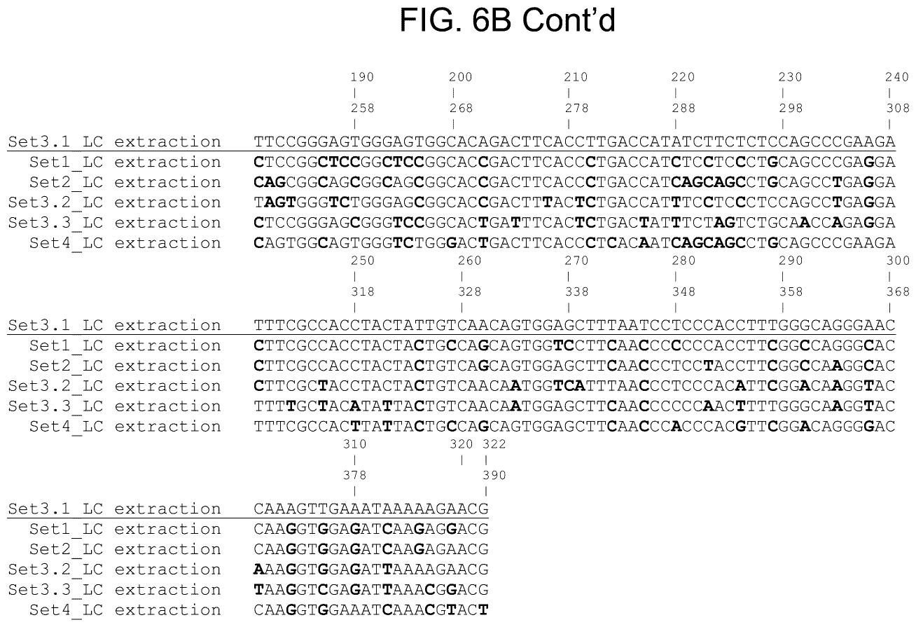

Surprisingly, as shown in FIG. 5, significant product titer differences were observed among the six codon sets. Codon sets obtained from Algorithm 1 (“Set 1”) and Algorithm 2 (“Set 2”), and one of the three codon sets from Algorithm 3 (Set 3.1, Set 3.2, Set 3.3) showed significantly higher titer than the others.

Table 2 summarizes the percent identity of different codon-optimized sequences.

TABLE 2

| Original Sequence | Codon Optimized Sequence | Percent Consensus |
|---|---|---|
| Set 3.1 HC | Set 1 HC | 93.5 |
| | Set 2 HC | 93.4 |
| | Set 3.2 HC | 93.7 |
| | Set 3.3 HC | 93.5 |
| | Set 4 HC | 93.6 |
| Set 3.1 LC | Set 1 LC | 89.8 |
| | Set 2 LC | 89.2 |
| | Set 3.2 LC | 88.9 |
| | Set 3.3 LC | 88.4 |
| | Set 4 LC | 89 |

In summary, the poor expression level of ocrelizumab in CHO host cells was solved by codon optimization. Among the different algorithms tested, three codon optimized sets showed 3-fold to 9-fold titer improvement from the original CHO GS-KO pools.

Example 7. Further Investigation into Codon Usage

Because certain sets of VH and VL codons significantly improved the expression level of ocrelizumab, further analyses on why certain codon sets perform better than others are conducted.

mRNA levels comparison study indicated that the mRNA level in high expression sets were much higher than low expression sets. This points towards the role of codons in transcription process. To understand the impact of individual codon, the first hypothesis is the assumption that there are some suicide codons that does not allow the transcription to move forward. We did not find this to be the case where some codons were responsible for stopping the transcription abruptly.

Every species has preferred codons that is used at higher frequency. We found from our data that the high expression sets use preferred codons at higher frequency compared to low expression sets. The following experiments are expected to further illustrate the roles of codon usage.

First, codon-based indices study is carried out. This study includes five parts. (1) Relative synonymous codon usage (computational). This part of the study is based on ratio between observed number of codons and number of times codon would be observed if the synonymous codon usage is completely random. The values for more frequent than average codon is greater than 1, less frequent codons have values less than 1, and average codons have a value of 1. (2) Codon preference bias (computational). This part of the study is based on multinomial and Poisson distributions. Higher value indicates more bias toward optimal codons. (3) The Scaled X (computational). This part of the study calculates deviation from equal usage of codon within the synonymous group divided by total number of codons in the gene using chi squared test. Higher value indicates a stronger bias. (4). Relative Codon Adaptation (computational). This part of the study compares observed and expected codon frequency. The results predict expression levels. Higher scores are attributed to genes that are more frequent in highly expressed genes. (5) RNA sequencing. Frozen cell pellets at D9 of FB are collected and whole RNA is extracted. Samples are used for RNA sequencing study using next generation sequencing. The results inform us tRNA availability and sequence dependent mRNA degradation. In addition, tRNA adaptation index computes weight for each codon based on tRNA copy number, which measures translation efficiency.

Additional tests related to codon bias indices are also available, all these tests can be conducted using suitable platforms such as Excel, MATLAB, Python, or R Studio.

References. Bahiri-Elitzur S, Tuller T. Codon-based indices for modeling gene expression and transcript evolution. Comput Struct Biotechnol J. 2021 Apr. 22;19:2646-2663. doi: 10.1016/j.csbj.2021.04.042. PMID: 34025951; PMCID: PMC8122159.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

TABLE A
sequence table

| SEQ | | |
|---|---|---|
| ID | | |
| 1 | ocrelizumab | DIQMTQSPSS LSASVGDRVT ITCRASSSVS YMHWYQQKPG KAPKPLIYAP |
| | light chain | SNLASGVPSR FSGSGSGTDF TLTISSLQPE DFATYYCQQW SFNPPTFGQG |
| | (LC) | TKVEIKRTVA APSVFIFPPS DEQLKSGTAS VVCLLNNFYP REAKVQWKVD |
| | | NALQSGNSQE SVTEQDSKDS TYSLSSTLTL SKADYEKHKV YACEVTHQGL |
| | | SSPVTKSENR GEC |
| | | (LC Kabat CDR1, CDR2, CDR3 underlined) |
| 2 | ocrelizumab | EVQLVESGGG LVQPGGSLRL SCAASGYTFT SYNMHWVRQA PGKGLEWVGA |
| | heavy chain | IYPGNGDTSY NQKFKGRFTI SVDKSKNTLY LQMNSLRAED TAVYYCARVV |
| | (HC) | YYSNSYWYFD VWGQGTLVTV SSASTKGPSV FPLAPSSKST SGGTAALGCL |
| | | VKDYFPEPVT VSWNSGALTS GVHTFPAVLQ SSGLYSLSSV VTVPSSSLGT |
| | | QTYICNVNHK PSNTKVDKKV EPKSCDKTHT CPPCPAPELL GGPSVFLFPP |
| | | KPKDTLMISR TPEVTCVVVD VSHEDPEVKF NWYVDGVEVH NAKTKPREEQ |
| | | YNSTYRVVSV LTVLHQDWLN GKEYKCKVSN KALPAPIEKT ISKAKGQPRE |
| | | PQVYTLPPSR EEMTKNQVSL TCLVKGFYPS SVMHEALHNH YTQKSLSLSP |
| | | PVLDSDGSFF LYSKLTVDKS RWQQGNVFSC DIAVEWESNG QPENNYKTTP |
| | | GK |
| | | (HC Kabat CDR1, CDR2, CDR3 underlined) |
| 3 | VL | DIQMTQSPSS LSASVGDRVT ITCRASSSVS YMHWYQQKPG KAPKPLIYAP |
| | | SNLASGVPSR FSGSGSGTDF TLTISSLQPE DFATYYCQQW SFNPPTFGQG |
| | | TKVEIK(R) |
| | | (LC Kabat CDR1, CDR2, CDR3 underlined) |
| | | junction residue ″R″ in parenthesis is optional. |
| 4 | VH | EVQLVESGGG LVQPGGSLRL SCAASGYTFT SYNMHWVRQA PGKGLEWVGA |
| | | IYPGNGDTSY NQKFKGRFTI SVDKSKNTLY LQMNSLRAED TAVYYCARVV |
| | | YYSNSYWYFD VWGQGTLVTV SS |
| | | (HC Kabat CDR1, CDR2, CDR3 underlined) |
| 5 | signal | ATGGACATGAGGGTGCCCGCTCAGCTCCTGGGGCTCCTGCTGCTGTGGCTGAGAGG |
| | peptide | TGCGCGCTGT |
| 6 | Set 1 | ATGGACATGAGGGTGCCCGCTCAGCTCCTGGGGCTCCTGCTGCTGTGGCTGAGAGG |
| | light chain | TGCGCGCTGTGACATCCAGATGACCCAGTCCCCCTCCTCCCTGTCCGCCTCCGTGG |
| | full length | GCGACAGGGTGACCATCACCTGCAGGGCCTCCTCCTCCGTGTCCTACATGCACTGG |
| | | TACCAACAGAAGCCTGGCAAGGCACCTAAGCCCCTGATCTACGCCCCTTCCAACTT |
| | | GGCCTCTGGCGTGCCTTCCAGGTTCTCCGGCTCCGGCTCCGGCACCGACTTCACCC |
| | | TGACCATCTCCTCCCTGCAGCCCGAGGACTTCGCCACCTACTACTGCCAGCAGTGG |
| | | TCCTTCAACCCCCCCACCTTCGGCCAGGGCACCAAGGTGGAGATCAAGAGGACGGT |
| | | GGCTGCACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAGCA |
| | | GGACAGCAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTGAGCTCG |
| | | CCCGTCACAAAGAGCTTCAACAGGGGAGAGTGTTAG |
| | | (signal peptide underlined; VL in bold) |
| 7 | Set 1 | GACATCCAGATGACCCAGTCCCCCTCCTCCCTGTCCGCCTCCGTGGGCGACAGGGT |
| | light chain | GACCATCACCTGCAGGGCCTCCTCCTCCGTGTCCTACATGCACTGGTACCAACAGA |
| | VL | AGCCTGGCAAGGCACCTAAGCCCCTGATCTACGCCCCTTCCAACTTGGCCTCTGGC |
| | | GTGCCTTCCAGGTTCTCCGGCTCCGGCTCCGGCACCGACTTCACCCTGACCATCTC |
| | | CTCCCTGCAGCCCGAGGACTTCGCCACCTACTACTGCCAGCAGTGGTCCTTCAACC |
| | | CCCCCACCTTCGGCCAGGGCACCAAGGTGGAGATCAAGAGG |
| 8 | Set 1 | ATGGACATGAGGGTGCCCGCTCAGCTCCTGGGGCTCCTGCTGCTGTGGCTGAGAGG |
| | heavy chain | TGCGCGCTGTGAGGTACAGCTGGTGGAGTCCGGCGGCGGCCTGGTGCAGCCTGGTG |
| | full length | GCAGTCTGAGGCTGTCCTGCGCCGCCTCCGGCTACACCTTCACCTCCTACAACATG |
| | | CACTGGGTGAGGCAGGCCCCCGGCAAGGGCCTGGAGTGGGTGGGCGCCATCTACCC |
| | | CGGCAACGGCGACACCTCCTACAACCAGAAGTTCAAGGGCAGGTTCACCATCTCCG |
| | | TGGACAAGTCCAAGAACACCCTGTACCTGCAGATGAACTCCCTGAGGGCCGAGGAC |
| | | ACCGCCGTGTACTACTGCGCCAGGGTGGTGTACTACTCCAACTCCTACTGGTACTT |
| | | CGACGTGTGGGGCCAGGGCACCCTGGTGACCGTGTCCTCCGCCTCCACCAAGGGCC |
| | | CTGGGCTGCCTGGTCAAGGACTACTTCCCCGAACCGGTGACGGTGTCGTGGAACTC |
| | | AGGCGCCCTGACCAGCGGCGTGCACACCTTCCCGGCTGTCCTACAGTCCTCAGGAC |
| | | TCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAAAGTTGA |
| | | GCCCAAATCTTGTGACAAAACTCACACATGCCCACCGTGCCCAGCACCTGAACTCC |
| | | TGGGGGGACCGTCAGTCTTCCTCTTCCCCCCAAAACCCAAGGACACCCTCATGATC |
| | | TCCCGGACCCCTGAGGTCACATGCGTGGTGGTGGACGTGAGCCACGAAGACCCTGA |
| | | GGTCAAGTTCAACTGGTACGTGGACGGCGTGGAGGTGCATAATGCCAAGACAAAGC |
| | | CGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTGTCCAACAAAGCCCT |
| | | CCCAGCCCCCATCGAGAAAACCATCTCCAAAGCCAAAGGGCAGCCCCGAGAACCAC |
| | | AGGTGTACACCCTGCCCCCATCCCGGGAGGAGATGACCAAGAACCAGGTCAGCCTG |
| | | ACCTGCCTGGTCAAAGGCTTCTATCCCAGCGACATCGCCGTGGAGTGGGAGAGCAA |
| | | TGGGCAGCCGGAGAACAACTACAAGACCACGCCTCCCGTGCTGGACTCCGACGGCT |
| | | CCTTCTTCCTCTATAGCAAGCTCACCGTGGACAAGAGCAGGTGGCAGCAGGGGAAC |
| | | GTCTTCTCATGCTCCGTGATGCATGAGGCTCTGCACAACCACTACACGCAGAAGAG |
| | | CCTCTCCCTGTCTCCGGGCAAATAG |
| | | (signal peptide underlined; VH in bold) |
| 9 | Set 1 | GAGGTACAGCTGGTGGAGTCCGGCGGCGGCCTGGTGCAGCCTGGTGGCAGTCTGAG |
| | heavy chain | GCTGTCCTGCGCCGCCTCCGGCTACACCTTCACCTCCTACAACATGCACTGGGTGA |
| | VH | GGCAGGCCCCCGGCAAGGGCCTGGAGTGGGTGGGCGCCATCTACCCCGGCAACGGC |
| | | GACACCTCCTACAACCAGAAGTTCAAGGGCAGGTTCACCATCTCCGTGGACAAGTC |
| | | CAAGAACACCCTGTACCTGCAGATGAACTCCCTGAGGGCCGAGGACACCGCCGTGT |
| | | ACTACTGCGCCAGGGTGGTGTACTACTCCAACTCCTACTGGTACTTCGACGTGTGG |
| | | GGCCAGGGCACCCTGGTGACCGTGTCCTCCGCCTCC |
| 10 | Set 2 | ATGGACATGAGGGTGCCCGCTCAGCTCCTGGGGCTCCTGCTGCTGTGGCTGAGAGG |
| | light chain | TGCGCGCTGTGACATTCAGATGACACAGAGCCCTAGCAGCCTGAGCGCTAGCGTGG |
| | full length | GCGACAGAGTGACCATCACCTGCAGAGCTAGCAGCAGCGTGAGCTACATGCACTGG |
| | | TATCAGCAGAAGCCTGGCAAGGCCCCTAAGCCTCTGATCTACGCCCCTAGCAACCT |
| | | GGCTAGCGGCGTGCCTAGCAGATTCAGCGGCAGCGGCAGCGGCACCGACTTCACCC |
| | | TGACCATCAGCAGCCTGCAGCCTGAGGACTTCGCCACCTACTACTGTCAGCAGTGG |
| | | AGCTTCAACCCTCCTACCTTCGGCCAAGGCACCAAGGTGGAGATCAAGAGAACGGT |
| | | GGCTGCACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAGCA |
| | | GGACAGCAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTGAGCTCG |
| | | CCCGTCACAAAGAGCTTCAACAGGGGAGAGTGTTAG |
| | | (signal peptide underlined; VL in bold) |
| 11 | Set 2 | GACATTCAGATGACACAGAGCCCTAGCAGCCTGAGCGCTAGCGTGGGCGACAGAGT |
| | light chain | GACCATCACCTGCAGAGCTAGCAGCAGCGTGAGCTACATGCACTGGTATCAGCAGA |
| | VL | AGCCTGGCAAGGCCCCTAAGCCTCTGATCTACGCCCCTAGCAACCTGGCTAGCGGC |
| | | GTGCCTAGCAGATTCAGCGGCAGCGGCAGCGGCACCGACTTCACCCTGACCATCAG |
| | | CAGCCTGCAGCCTGAGGACTTCGCCACCTACTACTGTCAGCAGTGGAGCTTCAACC |
| | | CTCCTACCTTCGGCCAAGGCACCAAGGTGGAGATCAAGAGA |
| 12 | Set 2 | ATGGACATGAGGGTGCCCGCTCAGCTCCTGGGGCTCCTGCTGCTGTGGCTGAGAGG |
| | heavy chain | TGCGCGCTGTGAGGTACAGCTGGTGGAGAGCGGCGGCGGCCTGGTGCAACCTGGTG |
| | full length | GCAGTCTGAGACTGAGCTGCGCCGCTAGCGGCTACACCTTCACGAGCTACAACATG |
| | | CACTGGGTGAGACAAGCCCCTGGCAAGGGCCTGGAGTGGGTGGGCGCCATCTACCC |
| | | TGGCAACGGCGACACAAGCTACAATCAGAAGTTCAAGGGCAGATTCACCATCAGCG |
| | | TGGACAAGAGCAAGAACACCCTGTACCTGCAGATGAACAGCCTGAGAGCCGAGGAC |
| | | ACCGCCGTGTACTACTGCGCTAGAGTGGTGTACTACAGCAACAGCTACTGGTACTT |
| | | CGACGTGTGGGGCCAAGGCACCCTGGTGACCGTGAGCAGCGCCTCCACCAAGGGCC |
| | | CATCGGTCTTCCCCCTGGCACCCTCCTCCAAGAGCACCTCTGGGGGCACAGCGGCC |
| | | CTGGGCTGCCTGGTCAAGGACTACTTCCCCGAACCGGTGACGGTGTCGTGGAACTC |
| | | AGGCGCCCTGACCAGCGGCGTGCACACCTTCCCGGCTGTCCTACAGTCCTCAGGAC |
| | | TCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAAAGTTGA |
| | | GCCCAAATCTTGTGACAAAACTCACACATGCCCACCGTGCCCAGCACCTGAACTCC |
| | | TGGGGGGACCGTCAGTCTTCCTCTTCCCCCCAAAACCCAAGGACACCCTCATGATC |
| | | TCCCGGACCCCTGAGGTCACATGCGTGGTGGTGGACGTGAGCCACGAAGACCCTGA |
| | | GGTCAAGTTCAACTGGTACGTGGACGGCGTGGAGGTGCATAATGCCAAGACAAAGC |
| | | CGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTGTCCAACAAAGCCCT |
| | | CCCAGCCCCCATCGAGAAAACCATCTCCAAAGCCAAAGGGCAGCCCCGAGAACCAC |
| | | AGGTGTACACCCTGCCCCCATCCCGGGAGGAGATGACCAAGAACCAGGTCAGCCTG |
| | | ACCTGCCTGGTCAAAGGCTTCTATCCCAGCGACATCGCCGTGGAGTGGGAGAGCAA |
| | | TGGGCAGCCGGAGAACAACTACAAGACCACGCCTCCCGTGCTGGACTCCGACGGCT |
| | | CCTTCTTCCTCTATAGCAAGCTCACCGTGGACAAGAGCAGGTGGCAGCAGGGGAAC |
| | | GTCTTCTCATGCTCCGTGATGCATGAGGCTCTGCACAACCACTACACGCAGAAGAG |
| | | CCTCTCCCTGTCTCCGGGCAAATAG |
| | | (signal peptide underlined; VH in bold) |
| 13 | Set 2 | GAGGTACAGCTGGTGGAGAGCGGCGGCGGCCTGGTGCAACCTGGTGGCAGTCTGAG |
| | heavy chain | ACTGAGCTGCGCCGCTAGCGGCTACACCTTCACGAGCTACAACATGCACTGGGTGA |
| | VH | GACAAGCCCCTGGCAAGGGCCTGGAGTGGGTGGGCGCCATCTACCCTGGCAACGGC |
| | | GACACAAGCTACAATCAGAAGTTCAAGGGCAGATTCACCATCAGCGTGGACAAGAG |
| | | CAAGAACACCCTGTACCTGCAGATGAACAGCCTGAGAGCCGAGGACACCGCCGTGT |
| | | ACTACTGCGCTAGAGTGGTGTACTACAGCAACAGCTACTGGTACTTCGACGTGTGG |
| | | GGCCAAGGCACCCTGGTGACCGTGAGCAGCGCCTCC |
| 14 | Set 3.1 | ATGGACATGAGGGTGCCCGCTCAGCTCCTGGGGCTCCTGCTGCTGTGGCTGAGAGG |
| | light chain | TGCGCGCTGTGACATCCAAATGACACAGTCCCCATCCAGTCTGTCCGCCTCTGTGG |
| | | GAGATCGGGTAACAATAACTTGCAGAGCATCTTCCTCTGTGAGCTACATGCACTGG |
| | | TATCAACAGAAACCAGGTAAGGCTCCTAAACCCCTGATTTATGCCCCAAGCAACTT |
| | | GGCAAGCGGAGTCCCTTCCAGATTTTCCGGGAGTGGGAGTGGCACAGACTTCACCT |
| | | TGACCATATCTTCTCTCCAGCCCGAAGATTTCGCCACCTACTATTGTCAACAGTGG |
| | | AGCTTTAATCCTCCCACCTTTGGGCAGGGAACCAAAGTTGAAATAAAAAGAACGGT |
| | | GGCTGCACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAGCA |
| | | GGACAGCAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTGAGCTCG |
| | | CCCGTCACAAAGAGCTTCAACAGGGGAGAGTGTTAG |
| | | (signal peptide underlined; VL in bold) |
| 15 | Set 3.1 VL | GACATCCAAATGACACAGTCCCCATCCAGTCTGTCCGCCTCTGTGGGAGATCGGGT |
| | | AACAATAACTTGCAGAGCATCTTCCTCTGTGAGCTACATGCACTGGTATCAACAGA |
| | | AACCAGGTAAGGCTCCTAAACCCCTGATTTATGCCCCAAGCAACTTGGCAAGCGGA |
| | | GTCCCTTCCAGATTTTCCGGGAGTGGGAGTGGCACAGACTTCACCTTGACCATATC |
| | | TTCTCTCCAGCCCGAAGATTTCGCCACCTACTATTGTCAACAGTGGAGCTTTAATC |
| | | CTCCCACCTTTGGGCAGGGAACCAAAGTTGAAATAAAAAGAA |
| 16 | Set 3.1 | ATGGACATGAGGGTGCCCGCTCAGCTCCTGGGGCTCCTGCTGCTGTGGCTGAGAGG |
| | heavy chain | TGCGCGCTGTGAGGTACAACTGGTCGAGTCAGGGGGCGGGCTGGTCCAGCCCGGTG |
| | | GTAGTTTGAGACTCTCATGTGCCGCAAGTGGATATACCTTTACTAGCTATAACATG |
| | | CATTGGGTCCGGCAGGCCCCTGGAAAGGGGTTGGAGTGGGTTGGTGCTATCTATCC |
| | | AGGGAACGGGGATACTAGCTACAACCAAAAATTCAAGGGACGGTTTACAATAAGTG |
| | | TTGATAAGTCTAAGAACACACTCTACCTCCAGATGAATAGTTTGCGGGCAGAAGAC |
| | | ACAGCTGTTTATTATTGTGCTCGTGTTGTCTACTACTCCAACAGTTACTGGTATTT |
| | | TGATGTGTGGGGACAAGGGACACTCGTCACAGTCTCTTCCGCCTCCACCAAGGGCC |
| | | CATCGGTCTTCCCCCTGGCACCCTCCTCCAAGAGCACCTCTGGGGGCACAGCGGCC |
| | | CTGGGCTGCCTGGTCAAGGACTACTTCCCCGAACCGGTGACGGTGTCGTGGAACTC |
| | | AGGCGCCCTGACCAGCGGCGTGCACACCTTCCCGGCTGTCCTACAGTCCTCAGGAC |
| | | TCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAAAGTTGA |
| | | GCCCAAATCTTGTGACAAAACTCACACATGCCCACCGTGCCCAGCACCTGAACTCC |
| | | TGGGGGGACCGTCAGTCTTCCTCTTCCCCCCAAAACCCAAGGACACCCTCATGATC |
| | | TCCCGGACCCCTGAGGTCACATGCGTGGTGGTGGACGTGAGCCACGAAGACCCTGA |
| | | GGTCAAGTTCAACTGGTACGTGGACGGCGTGGAGGTGCATAATGCCAAGACAAAGC |
| | | CGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTGTCCAACAAAGCCCT |
| | | CCCAGCCCCCATCGAGAAAACCATCTCCAAAGCCAAAGGGCAGCCCCGAGAACCAC |
| | | AGGTGTACACCCTGCCCCCATCCCGGGAGGAGATGACCAAGAACCAGGTCAGCCTG |
| | | ACCTGCCTGGTCAAAGGCTTCTATCCCAGCGACATCGCCGTGGAGTGGGAGAGCAA |
| | | TGGGCAGCCGGAGAACAACTACAAGACCACGCCTCCCGTGCTGGACTCCGACGGCT |
| | | CCTTCTTCCTCTATAGCAAGCTCACCGTGGACAAGAGCAGGTGGCAGCAGGGGAAC |
| | | GTCTTCTCATGCTCCGTGATGCATGAGGCTCTGCACAACCACTACACGCAGAAGAG |
| | | CCTCTCCCTGTCTCCGGGCAAATAG |
| | | (signal peptide underlined; VH in bold) |
| 17 | Set 3.1 VH | GAGGTACAACTGGTCGAGTCAGGGGGCGGGCTGGTCCAGCCCGGTGGTAGTTTGAG |
| | | ACTCTCATGTGCCGCAAGTGGATATACCTTTACTAGCTATAACATGCATTGGGTCC |
| | | GGCAGGCCCCTGGAAAGGGGTTGGAGTGGGTTGGTGCTATCTATCCAGGGAACGGG |
| | | GATACTAGCTACAACCAAAAATTCAAGGGACGGTTTACAATAAGTGTTGATAAGTC |
| | | TAAGAACACACTCTACCTCCAGATGAATAGTTTGCGGGCAGAAGACACAGCTGTTT |
| | | ATTATTGTGCTCGTGTTGTCTACTACTCCAACAGTTACTGGTATTTTGATGTGTGG |
| | | GGACAAGGGACACTCGTCACAGTCTCTTCCGCCTCC |
| 18 | Set 3.2 | ATGGACATGAGGGTGCCCGCTCAGCTCCTGGGGCTCCTGCTGCTGTGGCTGAGAGG |
| | light chain | TGCGCGCTGTGACATTCAGATGACCCAGTCACCCAGTTCACTGTCAGCTTCTGTTG |
| | | GGGACCGCGTAACAATCACCTGCCGGGCTAGTAGTTCTGTTAGCTACATGCATTGG |
| | | TATCAACAAAAGCCCGGTAAGGCACCAAAGCCTTTGATTTATGCACCTTCAAATCT |
| | | GGCAAGCGGAGTGCCCTCACGCTTTAGTGGGTCTGGGAGCGGCACCGACTTTACTC |
| | | TGACCATTTCCTCCCTCCAGCCTGAGGACTTCGCTACCTACTACTGTCAACAATGG |
| | | TCATTTAACCCTCCCACATTCGGACAAGGTACAAAGGTGGAGATTAAAAGAACGGT |
| | | GGCTGCACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAGCA |
| | | GGACAGCAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTGAGCTCG |
| | | CCCGTCACAAAGAGCTTCAACAGGGGAGAGTGTTAG |
| | | (signal peptide underlined; VL in bold) |
| 19 | Set 3.2 VL | GACATTCAGATGACCCAGTCACCCAGTTCACTGTCAGCTTCTGTTGGGGACCGCGT |
| | | AACAATCACCTGCCGGGCTAGTAGTTCTGTTAGCTACATGCATTGGTATCAACAAA |
| | | AGCCCGGTAAGGCACCAAAGCCTTTGATTTATGCACCTTCAAATCTGGCAAGCGGA |
| | | GTGCCCTCACGCTTTAGTGGGTCTGGGAGCGGCACCGACTTTACTCTGACCATTTC |
| | | CTCCCTCCAGCCTGAGGACTTCGCTACCTACTACTGTCAACAATGGTCATTTAACC |
| | | CTCCCACATTCGGACAAGGTACAAAGGTGGAGATTAAAAGA |
| 20 | Set 3.2 | ATGGACATGAGGGTGCCCGCTCAGCTCCTGGGGCTCCTGCTGCTGTGGCTGAGAGG |
| | heavy chain | TGCGCGCTGTGAGGTTCAACTGGTCGAGTCAGGCGGCGGTCTCGTCCAACCAGGAG |
| | | GCTCCCTGAGGCTGAGCTGTGCTGCCTCAGGATACACTTTCACATCATACAACATG |
| | | CACTGGGTGAGGCAGGCCCCCGGTAAGGGGCTGGAATGGGTAGGCGCAATCTACCC |
| | | AGGAAATGGCGACACATCTTATAACCAGAAATTTAAAGGAAGATTTACCATTTCTG |
| | | TAGATAAGTCTAAAAACACTCTTTACTTGCAGATGAACTCCTTGAGGGCTGAAGAC |
| | | ACCGCTGTGTACTACTGTGCCCGCGTTGTATACTATAGCAACAGTTACTGGTATTT |
| | | CGACGTTTGGGGGCAAGGAACTTTGGTCACAGTCTCATCCGCCTCCACCAAGGGCC |
| | | CATCGGTCTTCCCCCTGGCACCCTCCTCCAAGAGCACCTCTGGGGGCACAGCGGCC |
| | | CTGGGCTGCCTGGTCAAGGACTACTTCCCCGAACCGGTGACGGTGTCGTGGAACTC |
| | | AGGCGCCCTGACCAGCGGCGTGCACACCTTCCCGGCTGTCCTACAGTCCTCAGGAC |
| | | TCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAAAGTTGA |
| | | GCCCAAATCTTGTGACAAAACTCACACATGCCCACCGTGCCCAGCACCTGAACTCC |
| | | TGGGGGGACCGTCAGTCTTCCTCTTCCCCCCAAAACCCAAGGACACCCTCATGATC |
| | | TCCCGGACCCCTGAGGTCACATGCGTGGTGGTGGACGTGAGCCACGAAGACCCTGA |
| | | GGTCAAGTTCAACTGGTACGTGGACGGCGTGGAGGTGCATAATGCCAAGACAAAGC |
| | | CGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTGTCCAACAAAGCCCT |
| | | CCCAGCCCCCATCGAGAAAACCATCTCCAAAGCCAAAGGGCAGCCCCGAGAACCAC |
| | | AGGTGTACACCCTGCCCCCATCCCGGGAGGAGATGACCAAGAACCAGGTCAGCCTG |
| | | ACCTGCCTGGTCAAAGGCTTCTATCCCAGCGACATCGCCGTGGAGTGGGAGAGCAA |
| | | TGGGCAGCCGGAGAACAACTACAAGACCACGCCTCCCGTGCTGGACTCCGACGGCT |
| | | CCTTCTTCCTCTATAGCAAGCTCACCGTGGACAAGAGCAGGTGGCAGCAGGGGAAC |
| | | GTCTTCTCATGCTCCGTGATGCATGAGGCTCTGCACAACCACTACACGCAGAAGAG |
| | | CCTCTCCCTGTCTCCGGGCAAATAG |
| | | (signal peptide underlined; VH in bold) |
| 21 | Set 3.2 VH | GAGGTTCAACTGGTCGAGTCAGGCGGCGGTCTCGTCCAACCAGGAGGCTCCCTGAG |
| | | GCTGAGCTGTGCTGCCTCAGGATACACTTTCACATCATACAACATGCACTGGGTGA |
| | | GGCAGGCCCCCGGTAAGGGGCTGGAATGGGTAGGCGCAATCTACCCAGGAAATGGC |
| | | GACACATCTTATAACCAGAAATTTAAAGGAAGATTTACCATTTCTGTAGATAAGTC |
| | | TAAAAACACTCTTTACTTGCAGATGAACTCCTTGAGGGCTGAAGACACCGCTGTGT |
| | | ACTACTGTGCCCGCGTTGTATACTATAGCAACAGTTACTGGTATTTCGACGTTTGG |
| | | GGGCAAGGAACTTTGGTCACAGTCTCATCCGCCTCC |
| 22 | Set 3.3 | ATGGACATGAGGGTGCCCGCTCAGCTCCTGGGGCTCCTGCTGCTGTGGCTGAGAGG |
| | Light chain | TGCGCGCTGTGATATTCAGATGACTCAATCCCCCAGCAGCCTGTCTGCATCTGTAG |
| | | GAGATAGGGTTACTATCACTTGCAGAGCAAGTTCCTCAGTATCATATATGCACTGG |
| | | TATCAGCAAAAACCCGGCAAGGCTCCAAAGCCTCTTATCTACGCTCCTTCCAACCT |
| | | GGCTTCCGGCGTCCCCTCTAGATTCTCCGGGAGCGGGTCCGGCACTGATTTCACTC |
| | | TGACTATTTCTAGTCTGCAACCAGAGGATTTTGCTACATATTACTGTCAACAATGG |
| | | AGCTTCAACCCCCCAACTTTTGGGCAAGGTACTAAGGTCGAGATTAAACGGACGGT |
| | | GGCTGCACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAGCA |
| | | GGACAGCAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTGAGCTCG |
| | | CCCGTCACAAAGAGCTTCAACAGGGGAGAGTGTTAG |
| | | (signal peptide underlined; VL in bold) |
| 23 | Set 3.3 VL | GATATTCAGATGACTCAATCCCCCAGCAGCCTGTCTGCATCTGTAGGAGATAGGGT |
| | | TACTATCACTTGCAGAGCAAGTTCCTCAGTATCATATATGCACTGGTATCAGCAAA |
| | | AACCCGGCAAGGCTCCAAAGCCTCTTATCTACGCTCCTTCCAACCTGGCTTCCGGC |
| | | GTCCCCTCTAGATTCTCCGGGAGCGGGTCCGGCACTGATTTCACTCTGACTATTTC |
| | | TAGTCTGCAACCAGAGGATTTTGCTACATATTACTGTCAACAATGGAGCTTCAACC |
| | | CCCCAACTTTTGGGCAAGGTACTAAGGTCGAGATTAAACGG |
| 24 | Set 3.3 | ATGGACATGAGGGTGCCCGCTCAGCTCCTGGGGCTCCTGCTGCTGTGGCTGAGAGG |
| | heavy chain | TGCGCGCTGTGAGGTACAGCTTGTGGAGTCAGGCGGCGGTCTGGTTCAACCCGGTG |
| | | GCTCTCTGAGACTTAGCTGCGCTGCCTCCGGTTACACTTTTACATCTTACAACATG |
| | | CACTGGGTCCGCCAGGCCCCAGGAAAAGGACTTGAGTGGGTTGGTGCAATCTACCC |
| | | AGGAAACGGTGACACTTCTTACAACCAGAAGTTTAAGGGTAGATTCACCATCAGTG |
| | | TTGACAAATCCAAAAACACCCTCTATCTGCAAATGAACTCCCTCAGGGCAGAAGAC |
| | | ACCGCTGTGTACTACTGTGCCAGAGTAGTGTACTACTCTAATTCCTATTGGTACTT |
| | | CGATGTCTGGGGGCAAGGTACACTGGTGACCGTATCTTCCGCCTCCACCAAGGGCC |
| | | CATCGGTCTTCCCCCTGGCACCCTCCTCCAAGAGCACCTCTGGGGGCACAGCGGCC |
| | | CTGGGCTGCCTGGTCAAGGACTACTTCCCCGAACCGGTGACGGTGTCGTGGAACTC |
| | | AGGCGCCCTGACCAGCGGCGTGCACACCTTCCCGGCTGTCCTACAGTCCTCAGGAC |
| | | TCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAAAGTTGA |
| | | GCCCAAATCTTGTGACAAAACTCACACATGCCCACCGTGCCCAGCACCTGAACTCC |
| | | TGGGGGGACCGTCAGTCTTCCTCTTCCCCCCAAAACCCAAGGACACCCTCATGATC |
| | | TCCCGGACCCCTGAGGTCACATGCGTGGTGGTGGACGTGAGCCACGAAGACCCTGA |
| | | GGTCAAGTTCAACTGGTACGTGGACGGCGTGGAGGTGCATAATGCCAAGACAAAGC |
| | | CGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTGTCCAACAAAGCCCT |
| | | CCCAGCCCCCATCGAGAAAACCATCTCCAAAGCCAAAGGGCAGCCCCGAGAACCAC |
| | | AGGTGTACACCCTGCCCCCATCCCGGGAGGAGATGACCAAGAACCAGGTCAGCCTG |
| | | ACCTGCCTGGTCAAAGGCTTCTATCCCAGCGACATCGCCGTGGAGTGGGAGAGCAA |
| | | TGGGCAGCCGGAGAACAACTACAAGACCACGCCTCCCGTGCTGGACTCCGACGGCT |
| | | CCTTCTTCCTCTATAGCAAGCTCACCGTGGACAAGAGCAGGTGGCAGCAGGGGAAC |
| | | GTCTTCTCATGCTCCGTGATGCATGAGGCTCTGCACAACCACTACACGCAGAAGAG |
| | | CCTCTCCCTGTCTCCGGGCAAATAG |
| | | (signal peptide underlined; VH in bold) |
| 25 | Set 3.3 VH | GAGGTACAGCTTGTGGAGTCAGGCGGCGGTCTGGTTCAACCCGGTGGCTCTCTGAG |
| | | ACTTAGCTGCGCTGCCTCCGGTTACACTTTTACATCTTACAACATGCACTGGGTCC |
| | | GCCAGGCCCCAGGAAAAGGACTTGAGTGGGTTGGTGCAATCTACCCAGGAAACGGT |
| | | GACACTTCTTACAACCAGAAGTTTAAGGGTAGATTCACCATCAGTGTTGACAAATC |
| | | CAAAAACACCCTCTATCTGCAAATGAACTCCCTCAGGGCAGAAGACACCGCTGTGT |
| | | ACTACTGTGCCAGAGTAGTGTACTACTCTAATTCCTATTGGTACTTCGATGTCTGG |
| | | GGGCAAGGTACACTGGTGACCGTATCTTCCGCCTCC |
| 26 | Set 4 | ATGGACATGAGGGTGCCCGCTCAGCTCCTGGGGCTGCTGCTGCTGTGGCTGAGAGG |
| | Light chain | TGCGCGCTGTGACATTCAGATGACCCAGTCTCCAAGCAGCCTGTCTGCATCTGTGG |
| | | GGGACAGGGTCACAATCACTTGCAGGGCCAGCTCAAGTGTAAGTTACATGCACTGG |
| | | TATCAGCAGAAGCCAGGAAAGGCCCCCAAACCCCTGATTTATGCCCCCTCCAACCT |
| | | GGCTTCTGGAGTCCCTAGCCGCTTCAGTGGCAGTGGGTCTGGGACTGACTTCACCC |
| | | TCACAATCAGCAGCCTGCAGCCCGAAGATTTCGCCACTTATTACTGCCAGCAGTGG |
| | | AGCTTCAACCCACCCACGTTCGGACAGGGGACCAAGGTGGAAATCAAACGTACTGT |
| | | GGCTGCACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAGCA |
| | | GGACAGCAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTGAGCTCG |
| | | CCCGTCACAAAGAGCTTCAACAGGGGAGAGTGTTAG |
| | | (signal peptide underlined; VL in bold) |
| 27 | Set 4 VL | GACATTCAGATGACCCAGTCTCCAAGCAGCCTGTCTGCATCTGTGGGGGACAGGGT |
| | | CACAATCACTTGCAGGGCCAGCTCAAGTGTAAGTTACATGCACTGGTATCAGCAGA |
| | | AGCCAGGAAAGGCCCCCAAACCCCTGATTTATGCCCCCTCCAACCTGGCTTCTGGA |
| | | GTCCCTAGCCGCTTCAGTGGCAGTGGGTCTGGGACTGACTTCACCCTCACAATCAG |
| | | CAGCCTGCAGCCCGAAGATTTCGCCACTTATTACTGCCAGCAGTGGAGCTTCAACC |
| | | CACCCACGTTCGGACAGGGGACCAAGGTGGAAATCAAACGT |
| 28 | Set 4 heavy | ATGGACATGAGGGTGCCCGCTCAGCTCCTGGGGCTCCTGCTGCTGTGGCTGAGAGG |
| | chain | TGCGCGCTGTGAGGTACAACTGGTCGAGAGCGGGGGAGGGCTGGTGCAGCCTGGGG |
| | | GCTCACTGAGGCTGTCCTGCGCCGCTTCTGGCTACACATTTACCAGTTACAATATG |
| | | CACTGGGTACGGCAGGCCCCTGGTAAGGGCCTGGAATGGGTGGGAGCTATTTATCC |
| | | CGGAAATGGTGATACTTCCTACAATCAGAAGTTCAAAGGCAGGTTCACAATCAGCG |
| | | TGGACAAATCCAAGAACACACTGTACCTGCAGATGAACAGCCTGAGGGCCGAGGAC |
| | | ACCGCGGTCTATTACTGTGCAAGAGTGGTGTACTACAGCAACAGCTACTGGTACTT |
| | | CGACGTCTGGGGCCAGGGGACCCTGGTCACCGTCTCTAGCGCTAGCACCAAGGGCC |
| | | CATCGGTCTTCCCCCTGGCACCCTCCTCCAAGAGCACCTCTGGGGGCACAGCGGCC |
| | | CTGGGCTGCCTGGTCAAGGACTACTTCCCCGAACCGGTGACGGTGTCGTGGAACTC |
| | | AGGCGCCCTGACCAGCGGCGTGCACACCTTCCCGGCTGTCCTACAGTCCTCAGGAC |
| | | TCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAAAGTGGA |
| | | GCCCAAATCTTGTGACAAAACTCACACATGCCCACCGTGCCCAGCACCTGAACTCC |
| | | TGGGGGGACCGTCAGTCTTCCTCTTTCCCCCAAAACCCAAGGACACCCTCATGATC |
| | | TCCCGGACCCCTGAGGTCACATGCGTGGTGGTGGACGTGAGCCACGAAGACCCTGA |
| | | GGTCAAGTTCAACTGGTACGTGGACGGCGTGGAGGTGCATAATGCCAAGACAAAGC |
| | | CGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTCTCCAACAAAGCCCT |
| | | CCCAGCCCCCATCGAGAAAACCATCTCCAAAGCCAAAGGGCAGCCCCGAGAACCAC |
| | | AGGTGTACACCCTGCCCCCATCCCGGGAGGAGATGACCAAGAACCAGGTCAGCCTG |
| | | ACCTGCCTGGTCAAAGGCTTCTATCCCAGCGACATCGCCGTGGAGTGGGAGAGCAA |
| | | TGGGCAGCCGGAGAACAACTACAAGACCACGCCTCCCGTGCTGGACTCCGACGGCT |
| | | CCTTCTTCCTCTATAGCAAGCTCACCGTGGACAAGAGCAGGTGGCAGCAGGGGAAC |
| | | GTCTTCTCATGCTCCGTGATGCATGAGGCTCTGCACAACCACTACACGCAGAAGAG |
| | | CCTCTCCCTGTCTCCGGGCAAATAG |
| | | (signal peptide underlined; VH in bold) |
| 29 | Set 4 VH | GAGGTACAACTGGTCGAGAGCGGGGGAGGGCTGGTGCAGCCTGGGGGCTCACTGAG |
| | | GCTGTCCTGCGCCGCTTCTGGCTACACATTTACCAGTTACAATATGCACTGGGTAC |
| | | GGCAGGCCCCTGGTAAGGGCCTGGAATGGGTGGGAGCTATTTATCCCGGAAATGGT |
| | | GATACTTCCTACAATCAGAAGTTCAAAGGCAGGTTCACAATCAGCGTGGACAAATC |
| | | CAAGAACACACTGTACCTGCAGATGAACAGCCTGAGGGCCGAGGACACCGCGGTCT |
| | | ATTACTGTGCAAGAGTGGTGTACTACAGCAACAGCTACTGGTACTTCGACGTCTGG |
| | | GGCCAGGGGACCCTGGTCACCGTCTCTAGCGCTAGC |

1. A recombinant nucleic acid encoding an anti-CD20 antibody, wherein (i) said antibody comprises the light chain (LC) complementarity-determining region (CDR) 1, CDR2, and CDR3 of SEQ ID NO:3, and the heavy chain (HC) CDR1, CDR2, and CDR3 of SEQ ID NO: 4; and (ii) wherein said nucleic acid comprises codons that are optimized for Chinese hamster ovary (CHO) cell expression.

2. The nucleic acid of claim 1, wherein said antibody comprises a heavy chain variable region (VH) comprising SEQ ID NO. 4; and
a light chain variable region (VL) comprising SEQ ID NO. 3.

3. (canceled)

4. The nucleic acid of claim 1, wherein said antibody comprises a heavy chain comprising SEQ ID NO. 2; and a light chain comprising SEQ ID NO. 1.

5. (canceled)

6. The nucleic acid of claim 1, comprising a sequence that is at least 90% identical to SEQ ID NO. 7.

7. The nucleic acid of claim 1, comprising a sequence that is at least 90% identical to SEQ ID NO. 9.

8. The nucleic acid of claim 1, comprising a sequence that is at least 90% identical to SEQ ID NO. 11.

9. The nucleic acid of claim 1, comprising a sequence that is at least 90% identical to SEQ ID NO. 13.

10. The nucleic acid of claim 1, comprising a sequence that is at least 90% identical to SEQ ID NO. 15.

11. The nucleic acid of claim 1, comprising a sequence that is at least 90% identical to SEQ ID NO. 17.

12. The nucleic acid of claim 1, comprising a sequence that is at least 90% identical to SEQ ID NO. 19.

13. The nucleic acid of claim 1, comprising a sequence that is at least 90% identical to SEQ ID NO. 21.

14. The nucleic acid of claim 1, comprising a sequence that is at least 90% identical to SEQ ID NO. 23.

15. The nucleic acid of claim 1, comprising a sequence that is at least 90% identical to SEQ ID NO. 25.

16. The nucleic acid of claim 1, comprising a sequence that is at least 90% identical to SEQ ID NO. 27.

17. The nucleic acid of claim 1, comprising a sequence that is at least 90% identical to SEQ ID NO. 29.

18. (canceled)

19. A vector comprising the nucleic acid of claim 1.

20. A CHO cell comprising the nucleic acid of claim 1.

21. (canceled)

22. A method of making an anti-CD20 antibody, or antigen-binding fragment thereof, comprising culturing the host cell of claim 20 under a condition wherein said antibody or antigen-binding fragment is expressed by said host cell.

23. A recombinant nucleic acid comprising a nucleotide sequence selected from any one of SEQ ID NOs. 6-29.

24. A vector comprising the nucleic acid of claim 23.

25. A CHO cell comprising the nucleic acid of claim 23.


SEQUENCE LISTING (29 sequences)

SEQ ID NO: 1 - protein, 213 aa, synthetic construct
  REGION 1..213: note = Synthetic peptide
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
  REGION 1..452: note = Synthetic peptide
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 107 aa, synthetic construct
  REGION 1..107: note = Synthetic peptide
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 4 - protein, 122 aa, synthetic construct [sequence omitted]

SEQ ID NO: 5 - DNA, 66 bp, synthetic construct
  misc_feature 1..66: note = Synthetic peptide
  source 1..66: mol_type = other DNA
atggacatgagggtgcccgctcagctcctggggctcctgctgctgtggctgagaggtgcgcgctgt

SEQ ID NO: 6 - DNA, 708 bp, synthetic construct
  misc_feature 1..708: note = Synthetic peptide
  source 1..708: mol_type = other DNA
atggacatgagggtgcccgctcagctcctggggctcctgctgctgtggctgagaggtgcgcgctgtgacatccagatgacccagtccccctcctccctgtccgcctccgtgggcgacagggtgaccatcacctgcagggcctcctcctccgtgtcctacatgcactggtaccaacagaagcctggcaaggcacctaagcccctgatctacgccccttccaacttggcctctggcgtgccttccaggttctccggctccggctccggcaccgacttcaccctgaccatctcctccctgcagcccgaggacttcgccacctactactgccagcagtggtccttcaacccccccaccttcggccagggcaccaaggtggagatcaagaggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 7 - DNA, 321 bp, synthetic construct
  misc_feature 1..321: note = Synthetic peptide
  source 1..321: mol_type = other DNA
gacatccagatgacccagtccccctcctccctgtccgcctccgtgggcgacagggtgaccatcacctgcagggcctcctcctccgtgtcctacatgcactggtaccaacagaagcctggcaaggcacctaagcccctgatctacgccccttccaacttggcctctggcgtgccttccaggttctccggctccggctccggcaccgacttcaccctgaccatctcctccctgcagcccgaggacttcgccacctactactgccagcagtggtccttcaacccccccaccttcggccagggcaccaaggtggagatcaagagg

SEQ ID NO: 8 - DNA, 1425 bp, synthetic construct
  misc_feature 1..1425: note = Synthetic peptide
  source 1..1425: mol_type = other DNA
atggacatgagggtgcccgctcagctcctggggctcctgctgctgtggctgagaggtgcgcgctgtgaggtacagctggtggagtccggcggcggcctggtgcagcctggtggcagtctgaggctgtcctgcgccgcctccggctacaccttcacctcctacaacatgcactgggtgaggcaggcccccggcaagggcctggagtgggtgggcgccatctaccccggcaacggcgacacctcctacaaccagaagttcaagggcaggttcaccatctccgtggacaagtccaagaacaccctgtacctgcagatgaactccctgagggccgaggacaccgccgtgtactactgcgccagggtggtgtactactccaactcctactggtacttcgacgtgtggggccagggcaccctggtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaatag

SEQ ID NO: 9 - DNA, 372 bp, synthetic construct
  misc_feature 1..372: note = Synthetic peptide
  source 1..372: mol_type = other DNA
gaggtacagctggtggagtccggcggcggcctggtgcagcctggtggcagtctgaggctgtcctgcgccgcctccggctacaccttcacctcctacaacatgcactgggtgaggcaggcccccggcaagggcctggagtgggtgggcgccatctaccccggcaacggcgacacctcctacaaccagaagttcaagggcaggttcaccatctccgtggacaagtccaagaacaccctgtacctgcagatgaactccctgagggccgaggacaccgccgtgtactactgcgccagggtggtgtactactccaactcctactggtacttcgacgtgtggggccagggcaccctggtgaccgtgtcctccgcctcc

SEQ ID NO: 10 - DNA, 708 bp, synthetic construct
  misc_feature 1..708: note = Synthetic peptide
  source 1..708: mol_type = other DNA
atggacatgagggtgcccgctcagctcctggggctcctgctgctgtggctgagaggtgcgcgctgtgacattcagatgacacagagccctagcagcctgagcgctagcgtgggcgacagagtgaccatcacctgcagagctagcagcagcgtgagctacatgcactggtatcagcagaagcctggcaaggcccctaagcctctgatctacgcccctagcaacctggctagcggcgtgcctagcagattcagcggcagcggcagcggcaccgacttcaccctgaccatcagcagcctgcagcctgaggacttcgccacctactactgtcagcagtggagcttcaaccctcctaccttcggccaaggcaccaaggtggagatcaagagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - DNA, 321 bp, synthetic construct
  misc_feature 1..321: note = Synthetic peptide
  source 1..321: mol_type = other DNA
gacattcagatgacacagagccctagcagcctgagcgctagcgtgggcgacagagtgaccatcacctgcagagctagcagcagcgtgagctacatgcactggtatcagcagaagcctggcaaggcccctaagcctctgatctacgcccctagcaacctggctagcggcgtgcctagcagattcagcggcagcggcagcggcaccgacttcaccctgaccatcagcagcctgcagcctgaggacttcgccacctactactgtcagcagtggagcttcaaccctcctaccttcggccaaggcaccaaggtggagatcaagaga

SEQ ID NO: 12 - DNA, 1425 bp, synthetic construct
  misc_feature 1..1425: note = Synthetic peptide
  source 1..1425: mol_type = other DNA
atggacatgagggtgcccgctcagctcctggggctcctgctgctgtggctgagaggtgcgcgctgtgaggtacagctggtggagagcggcggcggcctggtgcaacctggtggcagtctgagactgagctgcgccgctagcggctacaccttcacgagctacaacatgcactgggtgagacaagcccctggcaagggcctggagtgggtgggcgccatctaccctggcaacggcgacacaagctacaatcagaagttcaagggcagattcaccatcagcgtggacaagagcaagaacaccctgtacctgcagatgaacagcctgagagccgaggacaccgccgtgtactactgcgctagagtggtgtactacagcaacagctactggtacttcgacgtgtggggccaaggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaatag

SEQ ID NO: 13 - DNA, 372 bp, synthetic construct
  misc_feature 1..372: note = Synthetic peptide
  source 1..372: mol_type = other DNA
gaggtacagctggtggagagcggcggcggcctggtgcaacctggtggcagtctgagactgagctgcgccgctagcggctacaccttcacgagctacaacatgcactgggtgagacaagcccctggcaagggcctggagtgggtgggcgccatctaccctggcaacggcgacacaagctacaatcagaagttcaagggcagattcaccatcagcgtggacaagagcaagaacaccctgtacctgcagatgaacagcctgagagccgaggacaccgccgtgtactactgcgctagagtggtgtactacagcaacagctactggtacttcgacgtgtggggccaaggcaccctggtgaccgtgagcagcgcctcc

SEQ ID NO: 14 - DNA, 708 bp, synthetic construct
  misc_feature 1..708: note = Synthetic peptide
  source 1..708: mol_type = other DNA
atggacatgagggtgcccgctcagctcctggggctcctgctgctgtggctgagaggtgcgcgctgtgacatccaaatgacacagtccccatccagtctgtccgcctctgtgggagatcgggtaacaataacttgcagagcatcttcctctgtgagctacatgcactggtatcaacagaaaccaggtaaggctcctaaacccctgatttatgccccaagcaacttggcaagcggagtcccttccagattttccgggagtgggagtggcacagacttcaccttgaccatatcttctctccagcccgaagatttcgccacctactattgtcaacagtggagctttaatcctcccacctttgggcagggaaccaaagttgaaataaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 15 - DNA, 322 bp, synthetic construct
  misc_feature 1..322: note = Synthetic peptide
  source 1..322: mol_type = other DNA
gacatccaaatgacacagtccccatccagtctgtccgcctctgtgggagatcgggtaacaataacttgcagagcatcttcctctgtgagctacatgcactggtatcaacagaaaccaggtaaggctcctaaacccctgatttatgccccaagcaacttggcaagcggagtcccttccagattttccgggagtgggagtggcacagacttcaccttgaccatatcttctctccagcccgaagatttcgccacctactattgtcaacagtggagctttaatcctcccacctttgggcagggaaccaaagttgaaataaaaagaa

SEQ ID NO: 16 - DNA, 1425 bp, synthetic construct
  misc_feature 1..1425: note = Synthetic peptide
  source 1..1425: mol_type = other DNA
atggacatgagggtgcccgctcagctcctggggctcctgctgctgtggctgagaggtgcgcgctgtgaggtacaactggtcgagtcagggggcgggctggtccagcccggtggtagtttgagactctcatgtgccgcaagtggatatacctttactagctataacatgcattgggtccggcaggcccctggaaaggggttggagtgggttggtgctatctatccagggaacggggatactagctacaaccaaaaattcaagggacggtttacaataagtgttgataagtctaagaacacactctacctccagatgaatagtttgcgggcagaagacacagctgtttattattgtgctcgtgttgtctactactccaacagttactggtattttgatgtgtggggacaagggacactcgtcacagtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaatag

SEQ ID NO: 17 - DNA, 372 bp, synthetic construct
  misc_feature 1..372: note = Synthetic peptide
  source 1..372: mol_type = other DNA
gaggtacaactggtcgagtcagggggcgggctggtccagcccggtggtagtttgagactctcatgtgccgcaagtggatatacctttactagctataacatgcattgggtccggcaggcccctggaaaggggttggagtgggttggtgctatctatccagggaacggggatactagctacaaccaaaaattcaagggacggtttacaataagtgttgataagtctaagaacacactctacctccagatgaatagtttgcgggcagaagacacagctgtttattattgtgctcgtgttgtctactactccaacagttactggtattttgatgtgtggggacaagggacactcgtcacagtctcttccgcctcc

SEQ ID NO: 18 - DNA, 708 bp, synthetic construct
  misc_feature 1..708: note = Synthetic peptide
  source 1..708: mol_type = other DNA
atggacatgagggtgcccgctcagctcctggggctcctgctgctgtggctgagaggtgcgcgctgtgacattcagatgacccagtcacccagttcactgtcagcttctgttggggaccgcgtaacaatcacctgccgggctagtagttctgttagctacatgcattggtatcaacaaaagcccggtaaggcaccaaagcctttgatttatgcaccttcaaatctggcaagcggagtgccctcacgctttagtgggtctgggagcggcaccgactttactctgaccatttcctccctccagcctgaggacttcgctacctactactgtcaacaatggtcatttaaccctcccacattcggacaaggtacaaaggtggagattaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 19 - DNA, 321 bp, synthetic construct
  misc_feature 1..321: note = Synthetic peptide
  source 1..321: mol_type = other DNA
gacattcagatgacccagtcacccagttcactgtcagcttctgttggggaccgcgtaacaatcacctgccgggctagtagttctgttagctacatgcattggtatcaacaaaagcccggtaaggcaccaaagcctttgatttatgcaccttcaaatctggcaagcggagtgccctcacgctttagtgggtctgggagcggcaccgactttactctgaccatttcctccctccagcctgaggacttcgctacctactactgtcaacaatggtcatttaaccctcccacattcggacaaggtacaaaggtggagattaaaaga

SEQ ID NO: 20 - DNA, 1425 bp, synthetic construct
  misc_feature 1..1425: note = Synthetic peptide
  source 1..1425: mol_type = other DNA
atggacatgagggtgcccgctcagctcctggggctcctgctgctgtggctgagaggtgcgcgctgtgaggttcaactggtcgagtcaggcggcggtctcgtccaaccaggaggctccctgaggctgagctgtgctgcctcaggatacactttcacatcatacaacatgcactgggtgaggcaggcccccggtaaggggctggaatgggtaggcgcaatctacccaggaaatggcgacacatcttataaccagaaatttaaaggaagatttaccatttctgtagataagtctaaaaacactctttacttgcagatgaactccttgagggctgaagacaccgctgtgtactactgtgcccgcgttgtatactatagcaacagttactggtatttcgacgtttgggggcaaggaactttggtcacagtctcatccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaatag

SEQ ID NO: 21 - DNA, 372 bp, synthetic construct
  misc_feature 1..372: note = Synthetic peptide
  source 1..372: mol_type = other DNA
gaggttcaactggtcgagtcaggcggcggtctcgtccaaccaggaggctccctgaggctgagctgtgctgcctcaggatacactttcacatcatacaacatgcactgggtgaggcaggcccccggtaaggggctggaatgggtaggcgcaatctacccaggaaatggcgacacatcttataaccagaaatttaaaggaagatttaccatttctgtagataagtctaaaaacactctttacttgcagatgaactccttgagggctgaagacaccgctgtgtactactgtgcccgcgttgtatactatagcaacagttactggtatttcgacgtttgggggcaaggaactttggtcacagtctcatccgcctcc

SEQ ID NO: 22 - DNA, 708 bp, synthetic construct
  misc_feature 1..708: note = Synthetic peptide
  source 1..708: mol_type = other DNA
atggacatgagggtgcccgctcagctcctggggctcctgctgctgtggctgagaggtgcgcgctgtgatattcagatgactcaatcccccagcagcctgtctgcatctgtaggagatagggttactatcacttgcagagcaagttcctcagtatcatatatgcactggtatcagcaaaaacccggcaaggctccaaagcctcttatctacgctccttccaacctggcttccggcgtcccctctagattctccgggagcgggtccggcactgatttcactctgactatttctagtctgcaaccagaggattttgctacatattactgtcaacaatggagcttcaaccccccaacttttgggcaaggtactaaggtcgagattaaacggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 23 - DNA, 321 bp, synthetic construct
  misc_feature 1..321: note = Synthetic peptide
  source 1..321: mol_type = other DNA
gatattcagatgactcaatcccccagcagcctgtctgcatctgtaggagatagggttactatcacttgcagagcaagttcctcagtatcatatatgcactggtatcagcaaaaacccggcaaggctccaaagcctcttatctacgctccttccaacctggcttccggcgtcccctctagattctccgggagcgggtccggcactgatttcactctgactatttctagtctgcaaccagaggattttgctacatattactgtcaacaatggagcttcaaccccccaacttttgggcaaggtactaaggtcgagattaaacgg

SEQ ID NO: 24 - DNA, 1425 bp, synthetic construct
  misc_feature 1..1425: note = Synthetic peptide
  source 1..1425: mol_type = other DNA
atggacatgagggtgcccgctcagctcctggggctcctgctgctgtggctgagaggtgcgcgctgtgaggtacagcttgtggagtcaggcggcggtctggttcaacccggtggctctctgagacttagctgcgctgcctccggttacacttttacatcttacaacatgcactgggtccgccaggccccaggaaaaggacttgagtgggttggtgcaatctacccaggaaacggtgacacttcttacaaccagaagtttaagggtagattcaccatcagtgttgacaaatccaaaaacaccctctatctgcaaatgaactccctcagggcagaagacaccgctgtgtactactgtgccagagtagtgtactactctaattcctattggtacttcgatgtctgggggcaaggtacactggtgaccgtatcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaatag

SEQ ID NO: 25 - DNA, 372 bp, synthetic construct
  misc_feature 1..372: note = Synthetic peptide
  source 1..372: mol_type = other DNA
gaggtacagcttgtggagtcaggcggcggtctggttcaacccggtggctctctgagacttagctgcgctgcctccggttacacttttacatcttacaacatgcactgggtccgccaggccccaggaaaaggacttgagtgggttggtgcaatctacccaggaaacggtgacacttcttacaaccagaagtttaagggtagattcaccatcagtgttgacaaatccaaaaacaccctctatctgcaaatgaactccctcagggcagaagacaccgctgtgtactactgtgccagagtagtgtactactctaattcctattggtacttcgatgtctgggggcaaggtacactggtgaccgtatcttccgcctcc

SEQ ID NO: 26 - DNA, 708 bp, synthetic construct
  misc_feature 1..708: note = Synthetic peptide
  source 1..708: mol_type = other DNA
atggacatgagggtgcccgctcagctcctggggctgctgctgctgtggctgagaggtgcgcgctgtgacattcagatgacccagtctccaagcagcctgtctgcatctgtgggggacagggtcacaatcacttgcagggccagctcaagtgtaagttacatgcactggtatcagcagaagccaggaaaggcccccaaacccctgatttatgccccctccaacctggcttctggagtccctagccgcttcagtggcagtgggtctgggactgacttcaccctcacaatcagcagcctgcagcccgaagatttcgccacttattactgccagcagtggagcttcaacccacccacgttcggacaggggaccaaggtggaaatcaaacgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 27 - DNA, 321 bp, synthetic construct
  misc_feature 1..321: note = Synthetic peptide
  source 1..321: mol_type = other DNA
gacattcagatgacccagtctccaagcagcctgtctgcatctgtgggggacagggtcacaatcacttgcagggccagctcaagtgtaagttacatgcactggtatcagcagaagccaggaaaggcccccaaacccctgatttatgccccctccaacctggcttctggagtccctagccgcttcagtggcagtgggtctgggactgacttcaccctcacaatcagcagcctgcagcccgaagatttcgccacttattactgccagcagtggagcttcaacccacccacgttcggacaggggaccaaggtggaaatcaaacgt

SEQ ID NO: 28 - DNA, 1425 bp, synthetic construct
  misc_feature 1..1425: note = Synthetic peptide
  source 1..1425: mol_type = other DNA
atggacatgagggtgcccgctcagctcctggggctcctgctgctgtggctgagaggtgcgcgctgtgaggtacaactggtcgagagcgggggagggctggtgcagcctgggggctcactgaggctgtcctgcgccgcttctggctacacatttaccagttacaatatgcactgggtacggcaggcccctggtaagggcctggaatgggtgggagctatttatcccggaaatggtgatacttcctacaatcagaagttcaaaggcaggttcacaatcagcgtggacaaatccaagaacacactgtacctgcagatgaacagcctgagggccgaggacaccgcggtctattactgtgcaagagtggtgtactacagcaacagctactggtacttcgacgtctggggccaggggaccctggtcaccgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcctggggggaccgtcagtcttcctctttcccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaatag

SEQ ID NO: 29 - DNA, 372 bp, synthetic construct
  misc_feature 1..372: note = Synthetic peptide
  source 1..372: mol_type = other DNA
gaggtacaactggtcgagagcgggggagggctggtgcagcctgggggctcactgaggctgtcctgcgccgcttctggctacacatttaccagttacaatatgcactgggtacggcaggcccctggtaagggcctggaatgggtgggagctatttatcccggaaatggtgatacttcctacaatcagaagttcaaaggcaggttcacaatcagcgtggacaaatccaagaacacactgtacctgcagatgaacagcctgagggccgaggacaccgcggtctattactgtgcaagagtggtgtactacagcaacagctactggtacttcgacgtctggggccaggggaccctggtcaccgtctctagcgctagc